(19) United States
(12) Patent Application Publication
(10) Pub. No.: US 2016/0074084 A1
(43) Pub. Date: Mar. 17, 2016

(54) WOVEN RETENTION DEVICES, SYSTEMS, PACKAGING, AND RELATED METHODS

(71) Applicant: Woven Orthopedic Technologies, LLC, Manchester, CT (US)

(72) Inventors: Christopher McDonnell, Sandy Hook, CT (US); Robert L. Richards, Hamden, CT (US); Nicole S. Sroka, Derby, CT (US); Ernest N. Corrao, JR., Bethel, CT (US); Ronald G. Litke, JR., Sandy Hook, CT (US)

(73) Assignee: Woven Orthopedic Technologies, LLC, Manchester, CT (US)

(21) Appl. No.: 14/856,355

(22) Filed: Sep. 16, 2015

Related U.S. Application Data
(60) Provisional application No. 62/051,095, filed on Sep. 16, 2014.

(51) Int. Cl. A61B 17/86

(52) U.S. Cl. CPC A61B 17/869; A61B 2017/0409

(57) ABSTRACT

A system for preparing and inserting a woven retention device into bone is provided. The system includes a woven retention device having a distal end, a proximal end for receiving a fastener, and a sleeve body between the distal and proximal ends. A longitudinal axis extends between the distal and proximal ends. The system also includes a measuring device surrounding at least a portion of the woven retention device and defining a cutting position at which the woven retention device is to be cut for insertion into a hole in the bone. The measuring device measures a distance from the distal end of the woven retention device to the cutting position. The woven retention device is movable relative to a least a portion of the measuring device that defines the cutting position such that the woven retention device can be cut to a desired size.

### CROSS-REFERENCE TO RELATED APPLICATIONS

This application claims priority to U.S. Provisional Application No. 62/051,095, filed Sep. 16, 2014, the disclosure of which is incorporated herein by reference in its entirety.

### TECHNICAL FIELD OF INVENTION

The present invention relates generally to woven retention device kits, systems, and methods for inserting a woven retention device into bone.

### BACKGROUND

In orthopedic surgery it is common to secure a bone screw to a patient's bone. Bone fracture repair is surgery to fix a broken bone using plates, nails, screws, or pins. It is common in the treatment of fractures to attach a plate to the bone utilizing bone screws. The resulting construct prevents motion of the fractured bone so that the bone can heal. Alternatively, one or more screws may be inserted across the break to hold it place.

In the treatment of spinal disorders, pedicle screws are inserted into the patient's vertebrae to serve as anchor points that can then be connected with a rod. This construct prevents motion of the vertebral segments that are to be fused.

In the treatment of detached tendons, screw-like tissue anchors are inserted into the patient's bone to serve as an anchor for the reattachment of the tendon.

One complication with the use of bone screws is the loss of fixation or grip between the bone screw and the patient's bone. Another complication with the use of bone screws is the stripping of the hole in the bone when the bone screw is inserted. This results in the loss of purchase and holding strength of the bone screw.

The presence of osteoporotic bone can increase the likelihood of complications by reducing the purchase or grip of the bone screw to the patient's bone, resulting in a loss of holding strength and loosening of the bone screw or pullout of the bone screw.

Current solutions to secure bone screws have not adequately addressed screw failure and the underlying causes of screw failure. In addition, there exists a need for simple and effective methods to insert such orthopedic devices and systems, as well as simple and easy to use kits that can enable surgeons to more effective treat patients.

### BRIEF SUMMARY OF THE INVENTION

According to an embodiment of the present invention, a system for preparing and inserting a woven retention device into bone is provided. The system includes a woven retention device having a distal end, a proximal end to receive a fastener, and a sleeve body between the distal and proximal ends. The sleeve body has a longitudinal axis extending between the distal and proximal ends. The system also includes a measuring device at least partially surrounding a portion of the woven retention device and defining a cutting position along the longitudinal axis. The cutting position is a position at which the woven retention device is to be cut for insertion into a hole in the bone. The measuring device is arranged to measure a distance from the distal end to the cutting position, and the woven retention device is movable relative to a least a portion of the measuring device that defines the cutting position such that the woven retention device can be cut to a desired size.

According to an embodiment, the system further includes a cutting implement to cut the woven retention device at the cutting position. The cutting implement can be coupled to the measuring device. The measuring device can include a hollow tube with a distal end and a proximal end, and an opening on the proximal end or the distal end of the hollow tube. The hollow tube is sized to surround at least a portion of the woven retention device, and the cutting implement can be integral with an interior of the hollow tube.

According to some embodiments, the measuring device includes a hollow tube with an opening on a proximal end or a distal end of the hollow tube, the hollow tube being sized to surround at least a portion of the woven retention device. The cutting position can be located at the proximal end or the distal end of the hollow tube. The hollow tube can include an opening around at least a portion of an outer circumferential surface of the hollow tube, the opening being located between the proximal and distal ends and defining the cutting position. The opening around the portion of the outer circumferential surface of the hollow tube can receive a cutting implement to cut the woven retention device, in some embodiments.

According to some embodiments, the system further includes the cutting implement, having a cutting edge able to cut the woven retention device through the opening. The cutting implement can be rotatable relative to the woven retention device and can cut the woven retention device while rotating relative to the woven retention device.

According to some embodiments, the measuring device includes one or more reference marks at fixed distances from the proximal or distal opening of the hollow tube, the one or more references marks indicating the distance from the distal end of the woven retention device to the cutting position. The hollow tube can include an opening or transparent portion on a circumferential wall of the hollow tube such that a position of the distal end of the woven retention device within the hollow tube relative to the one or more reference marks is visible to a user of the system from an exterior of the hollow tube.

According to some embodiments, the system further includes a push rod that is slideably received within the hollow tube and the woven retention device, and having a distal end to push the woven retention device through the distal opening of the hollow tube. The push rod can include one or more reference marks at predetermined distances from the distal end of the push rod, the reference marks being arranged to indicate the distance from the distal end of the woven retention device to the cutting position based on a relative position of the reference marks and the measuring device in a state where the push rod is slideably received within the hollow tube.

According to some embodiments, the measuring device includes a flexible band configured to surround the woven retention device at the cutting position. In some embodiments, the system further includes a rod for the woven retention device, the rod having a handle portion and a mounting portion, the mounting portion can be inserted within the woven retention device through the proximal end, and the rod can position the woven retention device, while mounted on the mounting portion, into the hole in the bone while the handle portion is held be a user of the system. The flexible band can be placed at the cutting position on the woven retention device while the woven retention device is mounted on the mounting portion and within the bone hole, the cutting position being approximately at an opening of the bone hole.

According to another embodiment, an apparatus for measuring and cutting a woven retention device for insertion into a bone is provided. The apparatus includes a hollow tube having a distal end with a distal opening and a proximal end with a proximal opening, the hollow tube being sized to contain the woven retention device in an interior of the hollow tube, and the distal opening being sized to pass the woven retention device therethrough. The apparatus also includes a cutting implement to cut the woven retention device to a desired length while at least a portion of the woven retention device is within the hollow tube. The apparatus further includes an advancement mechanism having a proximal rod that protrudes from the proximal end of the hollow tube, and a distal end to engage the woven retention device within the hollow tube. The advancement mechanism can change a position of the woven retention device within the hollow tube by sliding the woven retention device along a longitudinal axis of the hollow tube to increase or decrease the distance between a distal end of the woven retention device and the cutting implement. The advancement mechanism has reference marks indicating a distance from the distal end of the woven retention device to the cutting implement. The advancement mechanism can further include a user-operable lock for fixing a position of the distal end of the advancement mechanism.

### BRIEF DESCRIPTION OF DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1 shows an example of a woven retention device system being used to insert a woven retention devices into bone holes, according to an embodiment of the invention.

FIG. 2 shows the system of FIG. 1 comprising a woven retention device, a push rod, and a delivery tube, arranged as they may come in a package, according to an embodiment of the invention.

FIG. 3 shows the system of FIG. 2 where the woven retention device is being aligned with measuring indicia on the delivery tube to measure a desired length of the woven retention device.

FIG. 4A shows the system of FIG. 1 in a state where the push rod has been removed after measuring a desired length of the woven retention device but before cutting the woven retention deice.

FIG. 4B shows the system of FIG. 4A after the woven retention device is cut, leaving a portion of the woven retention device to be used in the delivery tube.

FIG. 5 shows the system of FIG. 4B where, after measuring and cutting the woven retention device, the push rod is reinserted into the delivery tube to push the woven retention device.

FIG. 6A shows the system of FIG. 5 where the woven retention device is being pushed out of the delivery tube by the push rod.

FIG. 6B shows the resulting woven retention device after emerging from the delivery tube in FIG. 6A.

FIGS. 7A-C show a system and method of using the system according to an embodiment, where FIGS. 7A, 7B, and 7C show the system in different stages of use according to the method.

FIG. 8A shows a system that uses a holder or collar around a woven retention device on a rod, according to an embodiment.

FIG. 8B shows the system of FIG. 8A after removal of the rod from the woven retention device.

FIG. 9A shows an embodiment of a system with a woven retention device provided on a rod.

FIG. 9B shows the system of FIG. 9A after cutting the woven retention device to a desired size, according to an embodiment.

FIG. 10 shows a system using a collar and measuring rod according to an embodiment.

FIG. 11 shows the system of FIG. 10 after cutting through the woven retention device and measuring rod.

FIG. 12 shows an enlarged cross-sectional view of the collar in FIG. 10, according to an embodiment.

FIG. 13 shows the collar and woven retention device of FIG. 10 after cutting the woven retention device and placing the woven retention device on the other end of the rod as compared to the view in FIG. 10.

FIG. 14 shows a cross-sectional view of the collar of FIG. 10, according to an embodiment.

FIG. 15 is a detailed view of a collar according to another embodiment.

FIG. 16 shows the collar of FIG. 15 used in a system, according to an embodiment of the invention.

FIG. 17 shows the system of FIG. 16 used with a cutting implement, according to an embodiment.

FIG. 18 shows a cross-sectional view of the collar and cutting implement of the system in FIG. 17, according to an embodiment.

FIGS. 19-22 show a system with an elastomer collar, according to an embodiment, with FIGS. 19, 20, 21, and 22 showing the system as used in different steps of a method of using the system.

FIG. 23 shows a system with a slidable measuring and cutting tool, according to an embodiment.

FIG. 24 shows a system where a measuring and cutting tool is provided on the packaging of the woven retention device, according to an embodiment.

FIG. 25 shows a device for measuring, cutting, and inserting a woven retention device, according to an embodiment.

FIG. 26 shows the device of FIG. 25 in a state where the woven retention device has been extended partially out of the device.

FIG. 27 shows a cross-sectional view of the device in FIG. 26, including a cross-sectional detail view to the distal end of the device and woven retention device.

FIG. 27A shows an enlarged view of the distal tip of the device in FIG. 27.

FIG. 28 shows a spring-loading apparatus with a woven retention device, according to an embodiment.

FIG. 29A shows the apparatus of FIG. 28 as it aligned with a bone hole prior to inserting a woven retention device, according to an embodiment.

FIG. 29B shows the apparatus of FIG. 28 as it measures a depth of a bone hole, according to an embodiment.

FIG. 29C shows an enlarged view of the markings on the device shown in FIG. 29B.

FIG. 30A shows the apparatus of FIG. 28 in which the spring-loaded woven retention device is in a position for receiving a push rod, according to an embodiment.

FIG. 30B shows the apparatus of FIG. 28 in which the woven retention device has been partially extended from the apparatus using the push rod.

FIG. 30C shows an enlarged view of the markings on the device shown in FIG. 30B.

FIG. 31A shows the apparatus of FIG. 28 in a state of cutting the woven retention device to a measured length, according to an embodiment.

FIG. 31B shows the measured and cut woven retention device of FIG. 31A being extended further from the apparatus for insertion into the bone hole.

FIG. 31C shows the apparatus of FIG. 28 in a state of inserting the woven retention device into the bone hole, according to an embodiment.

FIG. 31D shows an enlarged view of the markings on the device shown in FIG. 31B.

FIG. 32A shows a rotational cutting tool for a woven retention device, according to an embodiment.

FIG. 32B shows a cross-sectional view of the tool of FIG. 32A.

FIG. 33 shows a system for measuring, cutting, and/or inserting a woven retention device, according to an embodiment.

FIG. 34 shows the system of FIG. 33 as the woven retention device is being measured, according to an embodiment.

FIG. 35 shows the system of FIG. 33 after the woven retention device has been measured and cut using the system, according to an embodiment.

FIG. 36 shows the system of FIG. 33 as the woven retention device is pushed through the distal opening of the hollow tube and a separate view of the resulting woven retention device after being measured, cut, and having emerged from the hollow tube, according to an embodiment.

FIG. 37 shows a cutting implement used in a system according to an embodiment and a cross-sectional view of the system with the cutting implement for cutting a woven retention device.

### DETAILED DESCRIPTION OF THE INVENTION

It is to be understood that the following detailed description is exemplary and intended to provide further explanation without limiting the scope of the invention.

The devices, systems and methods described herein may be used in the area of orthopedics, for example, and, in particular, orthopedic repairs. These include various devices, systems and methods directed to fixing and/or retaining fasteners, preferably, in orthopedic applications and including, for example, cortical and/or bicortical bone. Fixing or retaining fasteners to bone tissue is complicated by the underlining bone tissue. Understanding that an underlying cause of failure with internal fixation in bone tissue is the bone, the devices, systems and methods described herein provide for solutions that address the implant site. At the implant site, the hole and the bone benefit from an enhanced interface.

The fixation and/or retention devices, systems and methods described herein maximize fixation and/or retention in the bone tissue, including, osteoporotic bone, bone of a poor quality, and mechanically poor bone in addition to healthy bone tissue. The fixation and/or retention devices, systems and methods described herein may be used with any type of fixation including any types of screws, pins, or other fasteners.

The devices, systems and methods described herein enhance the interaction of a bone anchor to a bone hole to provide enhanced fixation. Additionally, the devices, systems and methods may repair the surface of the bone hole following damage to the bone hole as in the case of stripping of the hole in the bone when a bone screw is over-tightened. Also, the devices, systems and methods provide for an enhanced bone hole surface for the reattachment of tendons in, for example, anterior/posterior cruciate ligament repair procedures, rotator cuff repair procedures, etc. The devices enhance the surface of a bone hole to enhance fixation of a bone anchor to bone and permits bone ingrowth into its structure. The devices enhance the interaction between the surface of a bone hole and the fixation device. The devices interdigitate with the bony structure and interact with the fixation device. The device alone, as a single device, enhances the surface of a bone hole to enhance fixation of a bone anchor to bone and accommodates variations in the diameter and depth of the bone hole. A person of ordinary skill in the art of orthopedic surgery would understand that aspects of the embodiments described herein can relate to mono- or unicortical screws and fixation, as well as bicortical screws and fixation. The devices, systems and methods can enhance fixation without requiring the use of cement and/or adhesives. However, according to some embodiments, cements and/or adhesive can be used in addition to or in place of another bone anchor such as a screw, pin, or other orthopedic implant.

Also described herein are methods of using these devices and systems, including methods of inserting fixation devices into a bone hole of a patient. The methods may use insertion tools, kits, and/or device or system packaging that aid a user of the fixation devices to achieve a desired placement and fit of the fixation device within the bone hole of the patient, in order to achieve optimal performance of the fixation device. Also described herein are kits or systems used for inserting fixation devices into a bone hole of a patient. The systems may include the fixation devices and tools for implanting the fixation devices. The systems may also include packaging of the fixation devices and tools.

According to some embodiments of the invention, systems, kits, and methods for inserting a woven retention device into a bone hole are provided that allow an operator of the systems, kits, and methods to achieve benefits such as maintaining visualization of the implant and implant site; securing the implant during operation; being able to quickly release the woven retention device into the implant site; performing a depth/length measurement, as well as cutting the woven retention device based on that depth/length measurement; providing a single use woven retention device that may be prepackaged tools for inserting the woven retention device and that may be pre-sized to fit different bone holes, or, alternatively, may configured to fit a variety of differently sized bone holes. In addition, the woven retention device may be measured and cut to a desired length corresponding to the length of the bone hole, or may be slightly greater than or less than the length of the bone hole. Embodiments of the invention provide solutions that are simple and easy to use with relatively low manufacturing costs.

As used herein, “cutting” or “cut” may refer to any action of separating one part of an element from another part of that element, including, for example, cutting, shearing, detaching, severing, disconnecting, melting, disintegrating, or dividing, and may be accomplished using any of a number of devices, processes, or methods, including, for example, physical, chemical, or thermal processes, scissors, scalpels, knives, punches, or any device, system or method capable of preforming any type of cutting or separating of an element into parts.

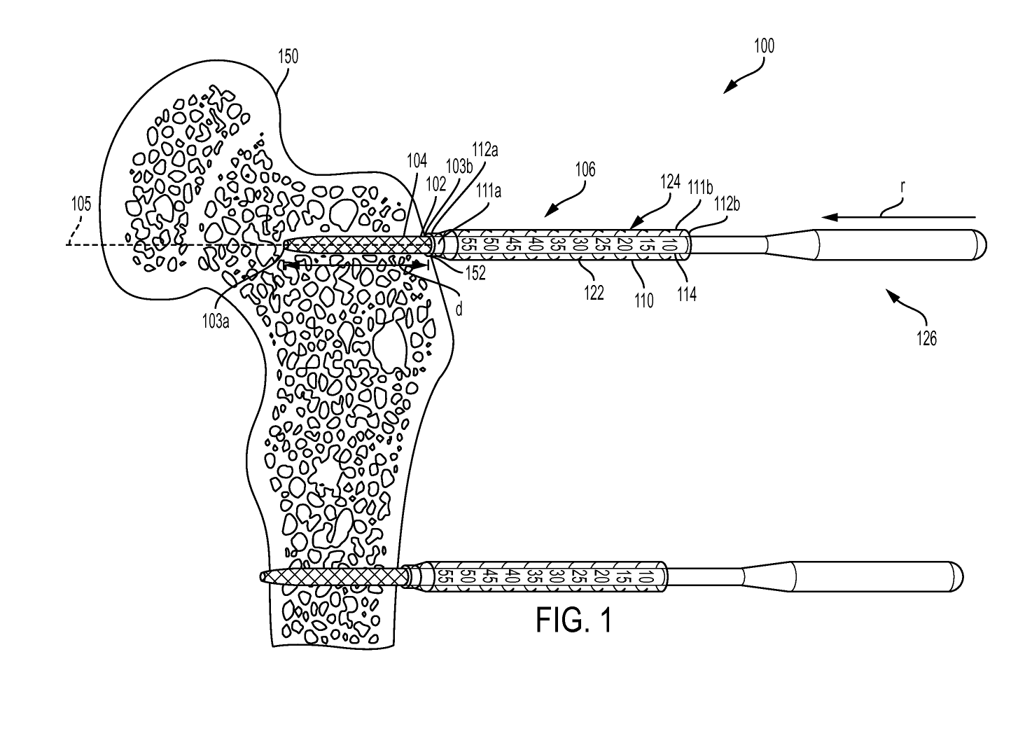

FIG. 1 shows an example of a system 100 used to insert a woven retention device 102 into a bone hole 152 in a bone 150, according to an embodiment. The system in FIG. 1 includes a woven retention device 102, a measurement device 106 comprising a hollow tube 110 , and a push rod 126 for pushing the woven retention device 102 through the hollow tube 110. The woven retention device 102 has a distal end 103a, a proximal end 103b sized and shaped to receive a fastener, and a sleeve body 104. The fastener (not pictured) can be a bone screw, pin, rod, or other implantable device, or soft tissue such as a tendon or artificial soft tissue, for example. The sleeve body 104 has a longitudinal axis 105, which, when implanted into the bone 150, can correspond to or be parallel with an insertion direction r in which the woven retention device 102 and push rod 126 are advanced toward and into the bone hole 152.

The hollow tube 110 of the measuring device 106 at least partially surround a portion of the woven retention device 102 during a procedure of inserting the woven retention device 102 into the bone 150. The measuring device 106 can define a cutting position 107 at a point along the longitudinal axis 105. The cutting position 107 is a predetermined position or positions at which the woven retention device 102 can be cut. In methods of using embodiments of the system, the woven retention device 102 can be cut to a desired size d corresponding to the depth of the bone hole 152. For example, the desired size d can be approximately equal to the depth of the bone hole 152 in some embodiments, or can be slightly longer than the depth of the bone hole 152 in a case, for example, where the woven retention device 102 is sized to extent through on or both sides of the bone, such as the bicortical positioning shown in the lower half of FIG. 1. The measuring device 106 is arranged to allow an operator of the system to measure a distance from the distal end 103a of the woven retention device 102 to the cutting position 107. In some embodiments, this measuring can be accomplished using reference marks 122 incorporated into the measuring device 106, for example. Reference marks 122 may communicate to an operator the distance from the distal end 103a to the cutting position 107 using specified units of measurement (e.g., millimeters) or may be reference marks without units of measure. According to embodiments, the woven retention device 102 is movable relative to the measuring device 106 so that the woven retention device can be measured and cut to the desired size d.

In the embodiment of FIG. 1, the reference marks 122 are arranged in ascending order from a proximal end 111b of the hollow tube 110 to a distal end 111a of the hollow tube 110. Thus, the distal end 103a of the woven retention device 102 can be placed at the reference mark 122 indicated the desired size d of the woven retention device 102 for implantation. Then, when cut the cutting position 107 at a proximal opening 112b of the hollow tube 110, the woven retention device will be cut to the desired size. The positioning of the woven retention device 102 can be achieved using the push rod 126 which has a distal end that push on or near the distal end 103a of the woven retention device. After being cut to the desired size d, the woven retention device 102 can be pushed by the push rod 126 through a distal opening 112a of the hollow tube 110 and into the bone hole 152.

According to embodiments of the system, a surgeon or user of the system can manually cut (using, for example, operating room scissors or other cutting implement) the woven retention device 102 to a desired length d based on the size of the bone hole 152, or the system may be provided as an all-in-one solution that incorporates cutting into the system itself. Embodiments of both types will be discussed below.

The embodiment in FIG. 1 is an example of an embodiment involving a manual cutting of the woven retention device. A cutting implement is not shown in FIG. 1, but scissors or another cutting instrument can be used.

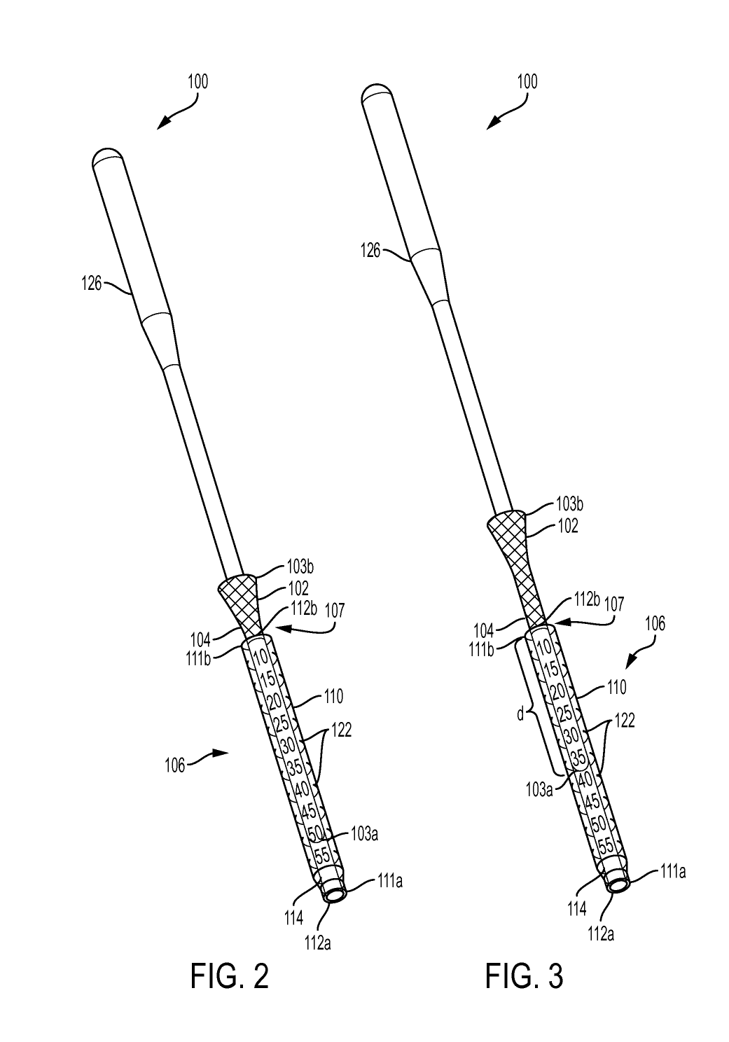

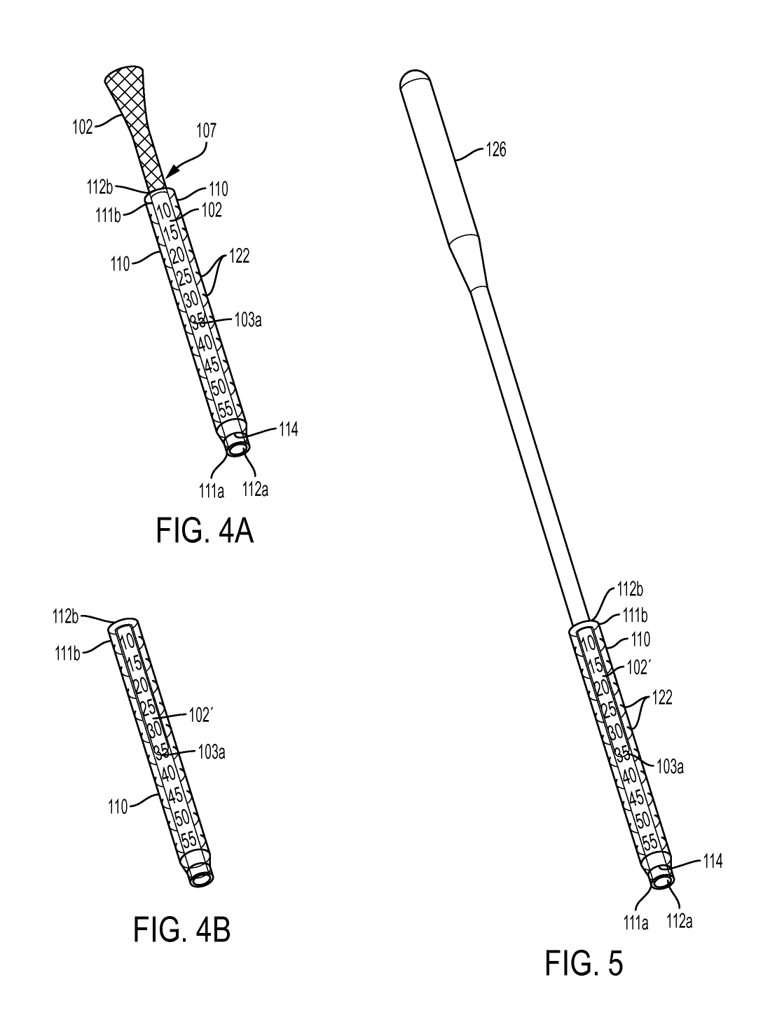

FIG. 2 shows a close-up view of the system 100 shown in FIG. 1. The system 100 may include packaging (not shown) in which the components of the system are prearranged in the arrangement shown in FIG. 2. For example, the woven retention device 102 may come pre-arranged at least partially within the hollow tube 110 and the push rod 126 may come pre-arranged at least partially inserted into the woven retention device 102. In this way, the system 100 is ready to use without requiring assembly by the user. However, the components of the system 100 may also be arranged separately or even packaged separately and then arranged by the user in the arrangement shown in FIG. 2. In this embodiment, the hollow tube 110 is made of a transparent material so that the distal end 103a of the woven retention device 102 can be seen relative to the reference marks 122. In FIG. 3, the position of the woven retention device 102 is adjusted relative to the reference marks 122 on the clear body of the hollow tube 110. Specifically, the woven retention device 102 is moved away from the distal end 111a of the hollow tube 110 relative to FIG. 2, so that the desired size d can be achieved after cutting the woven retention device 102 at the cutting position 107. After being placed in the position shown in FIG. 3, the push rod 126 can be removed, as shown in FIG. 4A, for cutting the woven retention device 102. After cutting, a woven retention device 102′ having the desired size d can remain inside the hollow tube 110, as shown in FIG. 4B, so that it is ready to be implanted into the bone 150.

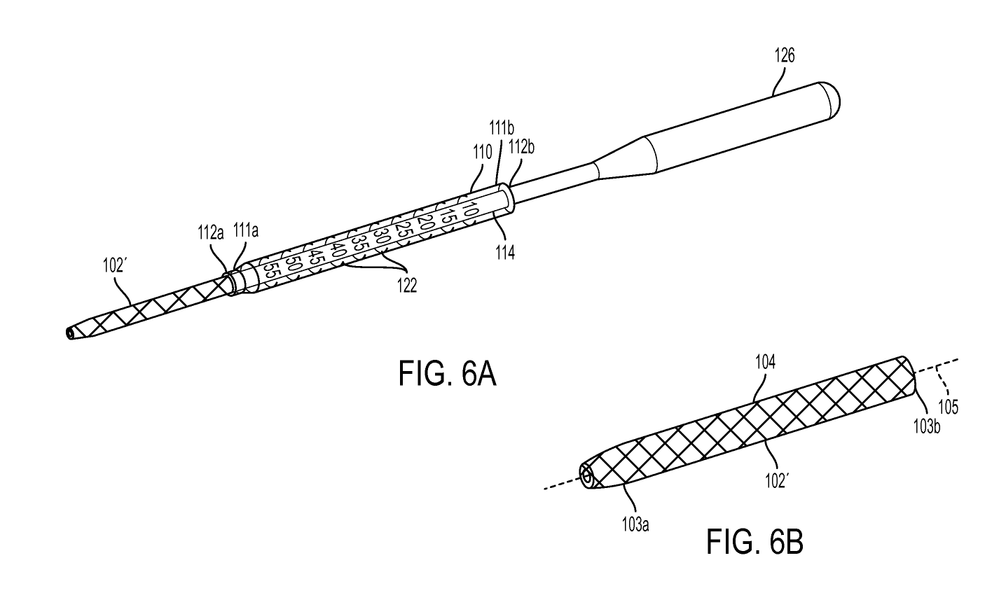

Then, as shown in FIG. 5, the push rod 126 can be inserted into the woven retention device 102′ through the proximal end 103b of the woven retention device 102′. The push rod 126 is sized and shaped to be able to push the woven retention device 102′ through the distal opening 112a of the hollow tube 110 for placement in the bone. FIG. 6A shows an example of the woven retention device 102′ emerging from the distal end 111a of the hollow tube 110. FIG. 6B shows a close-up view of the woven retention device 102′ after emerging from the hollow tube 110, according to an embodiment. After the woven retention device 102′ emerges from the hollow tube 110, the hollow tube 110 and push rod 126 can be withdrawn, leaving behind the lone woven retention device 102′, shown in FIG. 6B, in the bone hole (bone hole not shown in FIG. 6B). Subsequently, a fastener such as a screw can be inserted into the woven retention device 102′ in the bone hole 152. Alternatively, in some embodiments, the fastener may be provided within the woven retention device before insertion of the woven retention device into the bone hole. It is noted that the woven structure of woven retention devices 102 and 102′ is shown schematically in the drawings and not in detail. The woven structure can be arranged in various ways, according to different embodiments.

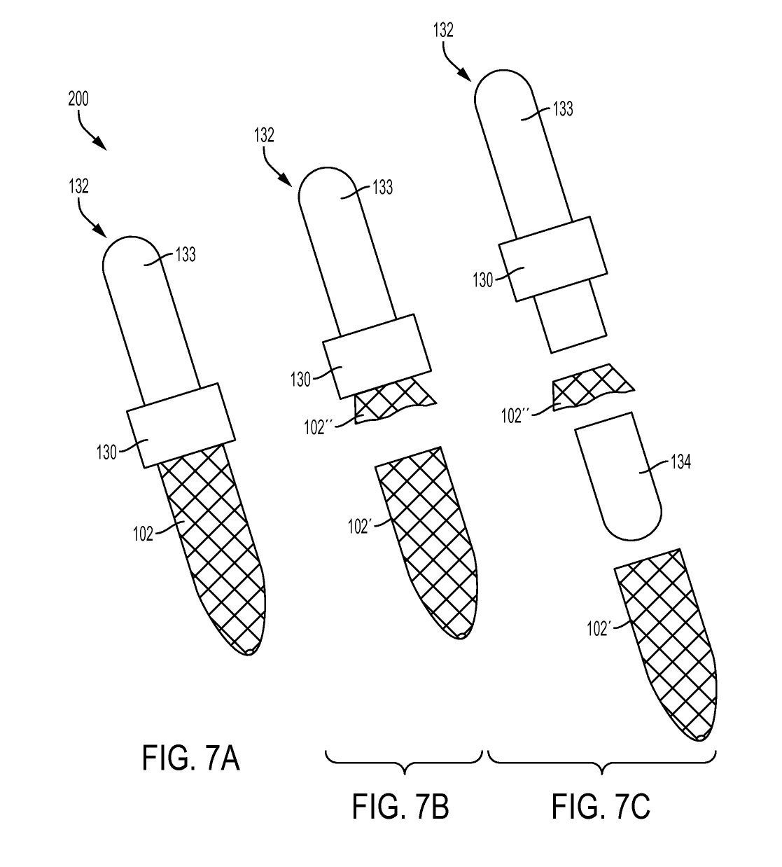

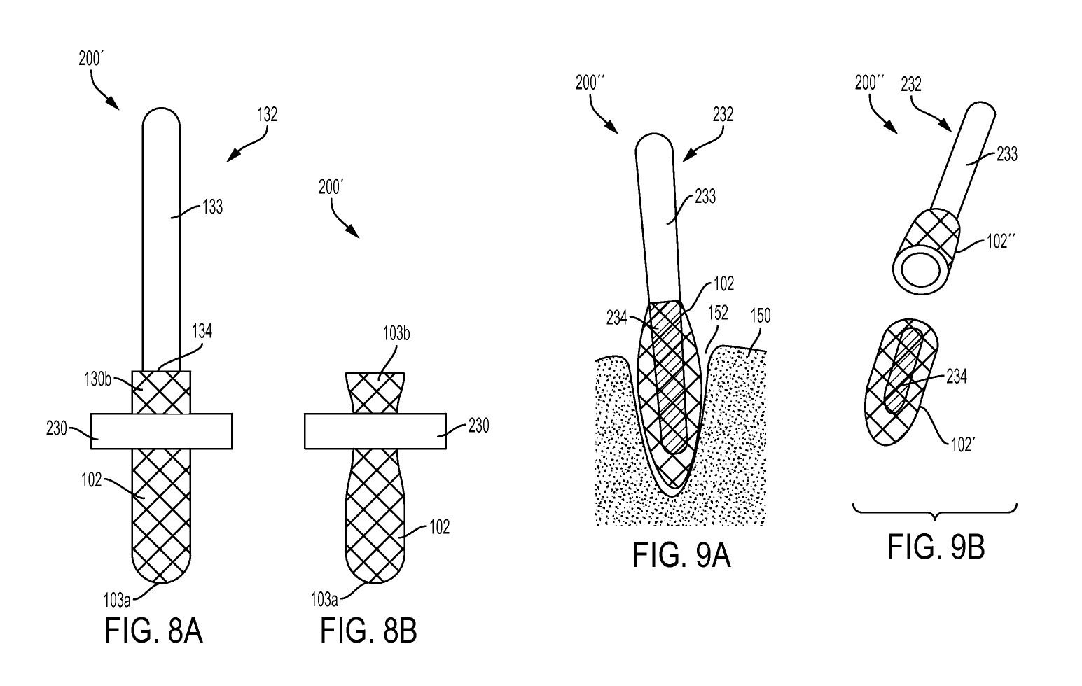

FIGS. 7A-7C show a system 200 according to an embodiment of the invention. In FIG. 7A, the woven retention device 102 is provided on a mounting portion 134 of a rod 132, and a proximal end of the woven retention device 102 is held on the rod 132 by a ring or collar 130. The rod 132 may be constructed to be cut by a cutting implement when cutting the woven retention device 102. For example, when a desired length of the woven retention device 102 is cut, the rod 132 can be cut through, which may ease the cutting process, in some embodiments. The rod 132 may be a rod for inserting the woven retention device 102 into a bone hole, or may be used solely for providing and preparing the woven retention device before insertion into the bone. FIGS. 8 and 9 show other examples according to variations of this embodiment.

In FIGS. 8A and 8B, the system 200′ includes a collar 230 acting as a measuring device and surrounding part of the woven retention device 102. The collar 230 can be placed on the woven retention device 102 such that a cutting position is defined on either side of the collar 230. The collar 230 may have some grip or elasticity such that it remains in place on the woven retention device even when the rod 132 is removed, as shown in FIG. 8B. Thus, the woven retention device 102 can be cut without cutting through the rod 132. In some embodiments, a user of system 200′ can position the collar 230 while the woven retention device 102 is placed within a bone hole while the user holds the handle portion 133. Then, once the collar 230 is in position, the user can lift the rod 132 and woven retention device 102 out of the bone hole, and optionally remove the rod 132 before cutting the woven retention device 102. The system 200″ of FIGS. 9A and 9B can be used similarly. However, as shown in FIG. 9B, the rod 232 can be cut when cutting the woven retention device.

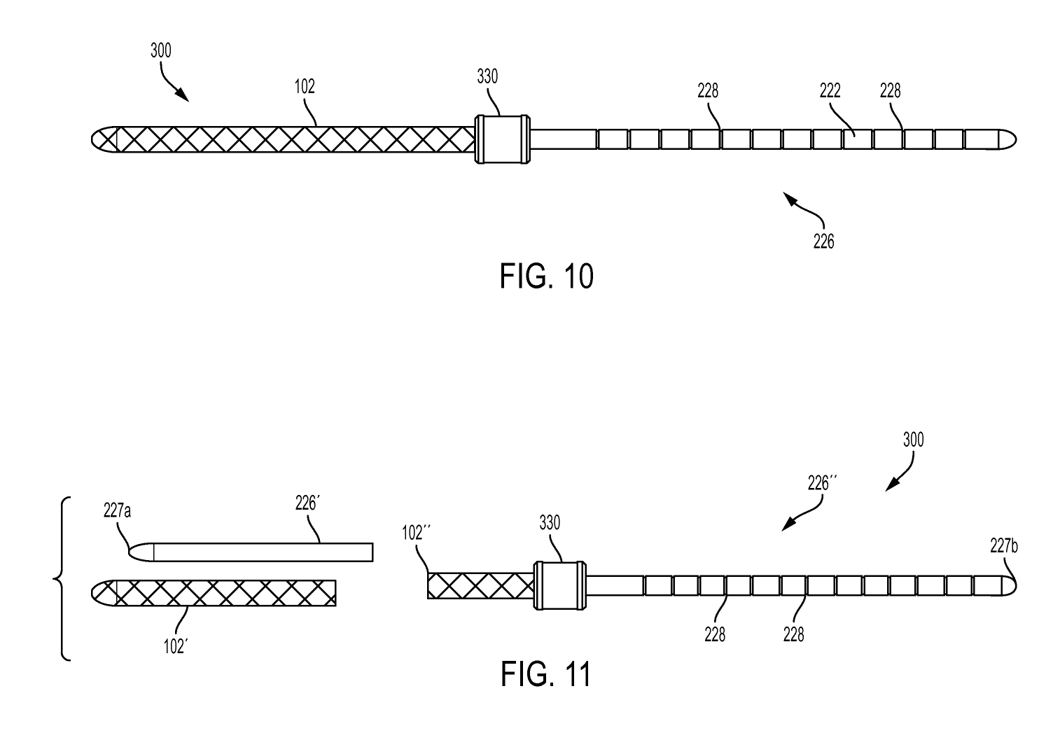

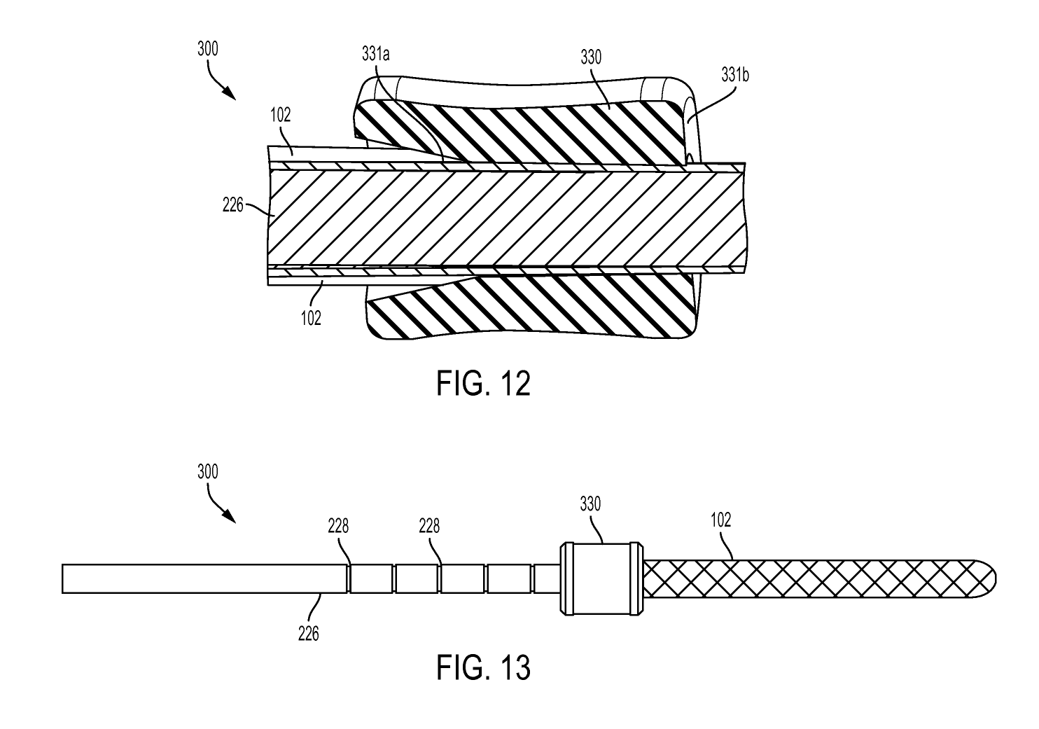

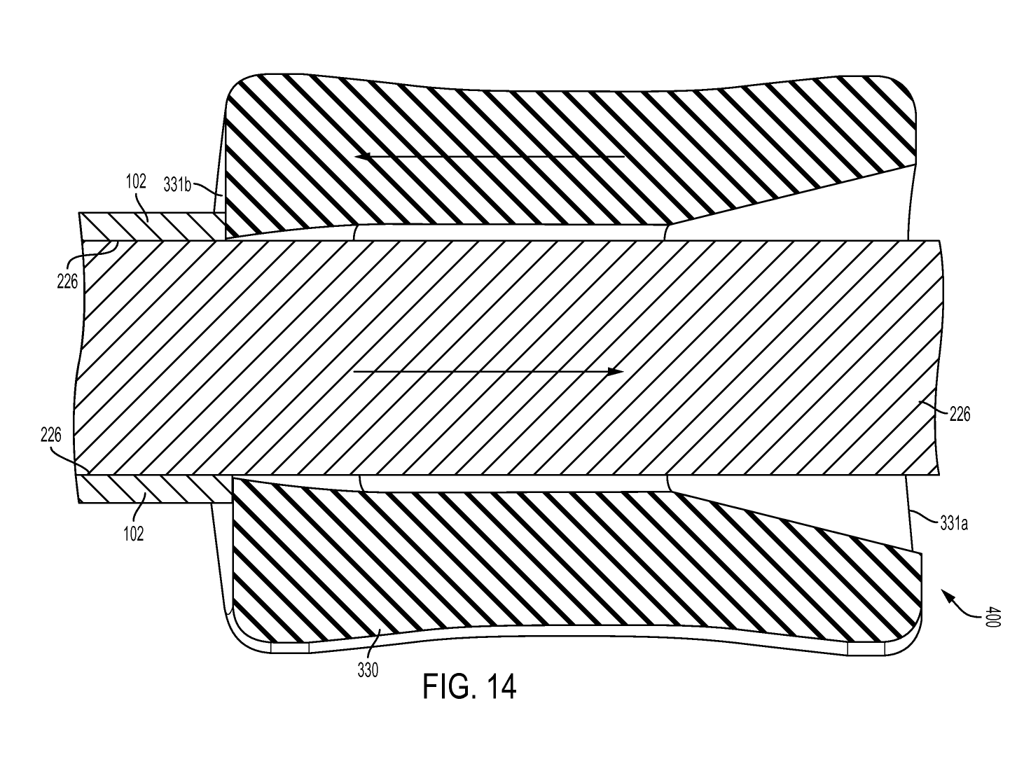

FIG. 10 shows an embodiment of a system 300 in which the woven retention device 102 is provided on a rod 226, optionally having witness marks or measuring indices 228 on the rod 226, and held on the rod 226 with a collar 330, arranged as a hollow tube. The collar 330 and witness marks or measuring indices 228 can together form a measuring device for measuring the woven retention device 102, in some embodiments. For example, the witness marks or measuring indices 228 on the rod 226 can be used as a depth gauge to measure the depth of a bone hole, in one embodiment. After obtaining the depth of the bone hole, the rod 226 can be removed and the woven retention device 102 cut using scissors or another suitable cutting implement. In some embodiments, the collar 330 can be moved to the cutting position. In some embodiments, the collar 330 may include a cutting implement in an interior of the collar 330. Optionally, the rod 226 may be cut through when cutting the woven retention device 102, as shown in FIG. 11. The cut end 226′ having the distal end 227a may then be disposed and the woven retention device 102′ inserted into the bone. Optionally, to place the woven retention device 102′ into the bone hole, the woven retention device 102′ can be mounted on a remaining portion of the rod 226″, including the proximal end 227b. FIG. 12 is a close-up view of the collar 330, which holds the woven retention device 102 in close contact with the rod 226. After cutting the woven retention device 102 to a desired length, the woven retention device 102′ may be placed on the opposite end of the rod 226, as shown in FIG. 13, for inserting into the bone hole. In the position shown in FIG. 13, the collar 330 is used to hold the woven retention device 102 on the rod 226 during the insertion process, according to some embodiments. When the woven retention device 102′ is in the desired position in the bone, the rod 226 can be removed to leave the woven retention device 102′ behind by pulling the rod 226 out (in a proximal direction, as shown by the black arrow in FIG. 14), while applying distally-directed pressure on the collar (shown by the white arrow in FIG. 14). By applying downward or distal pressure on the collar, the woven retention device can be held in place as the inserter stick is removed. As shown in FIG. 14, an inner surface of the collar 330 can be contoured to allow removal of the rod 226 while keeping the woven retention device 102 in place.

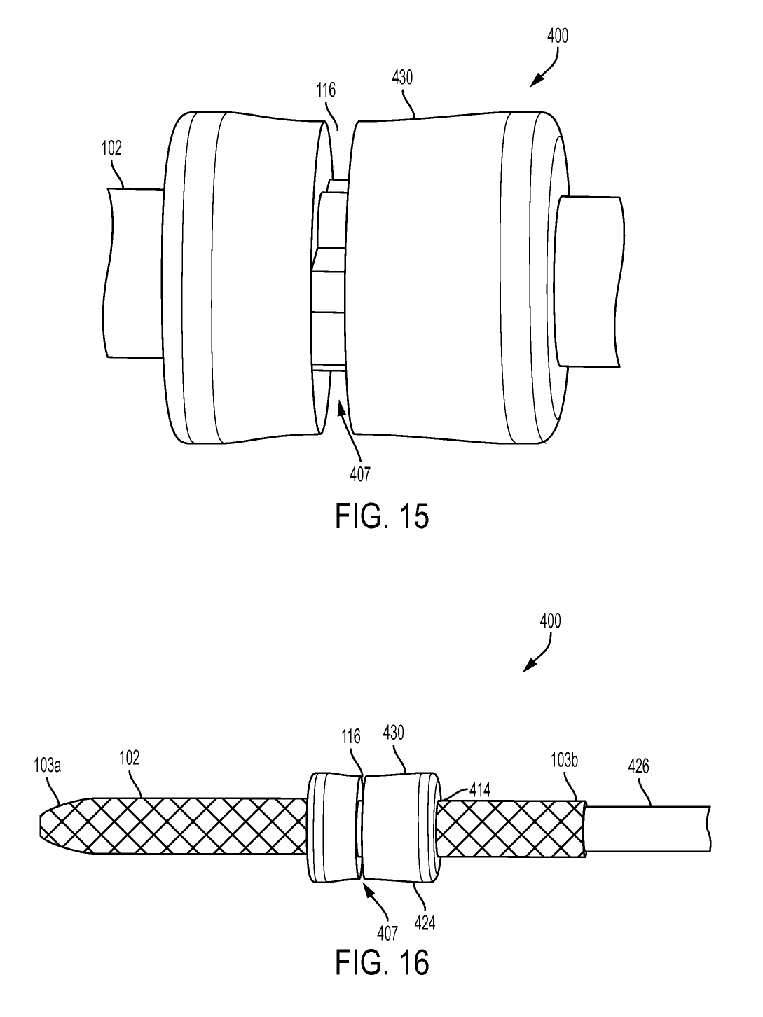

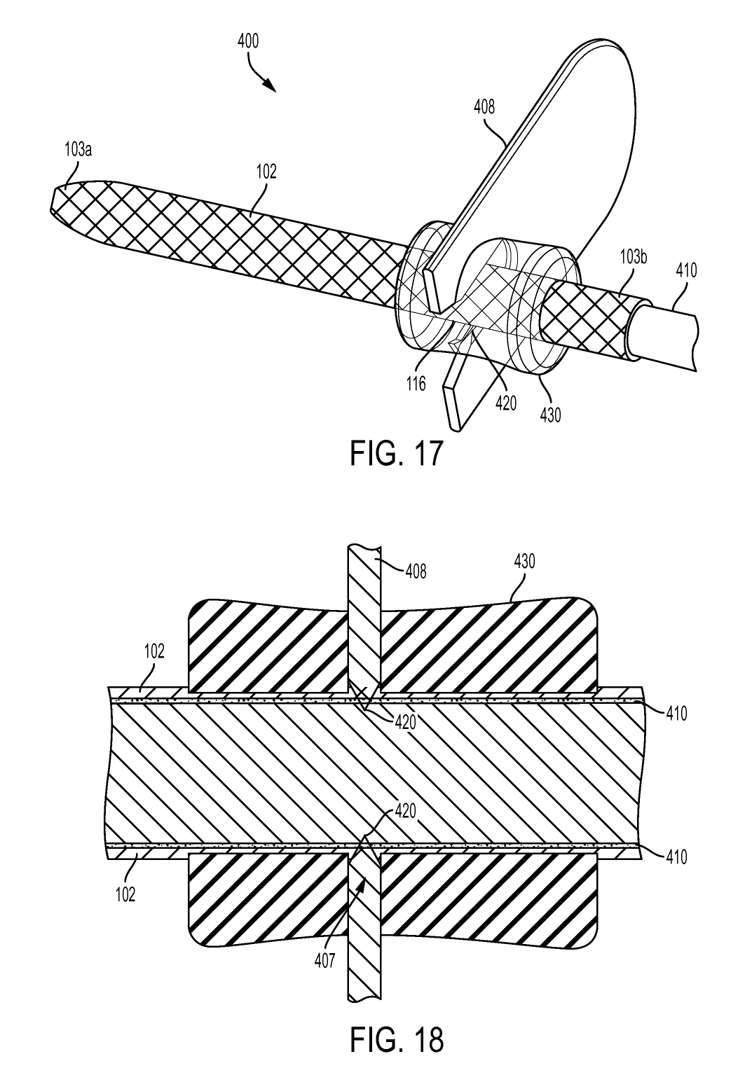

According to some embodiments, a system 400 can include a collar 430 with a space or slot 116 formed in the collar 430 to allow for cutting of the woven retention device 102, as shown in FIGS. 15 and 16. Thus, the slot 116 is positioned at the desired cutting position 407 of the woven retention device 102. The collar 430 may be used with or without a rod 426. The system 400 may also include a specifically designed cutting tool 408, such as that shown in FIG. 17, for example. As shown in FIG. 18, a cutting edge 420 of the cutting tool 408 fits within the slot 116 formed in the collar 430 and cuts the woven retention device 102. This cutting process may involve rotation of one or both of the collar 430 and cutting tool 408 relative to the woven retention device 102. Alternatively, other cutting implements such as scissors or scalpels maybe used to cut the woven retention device.

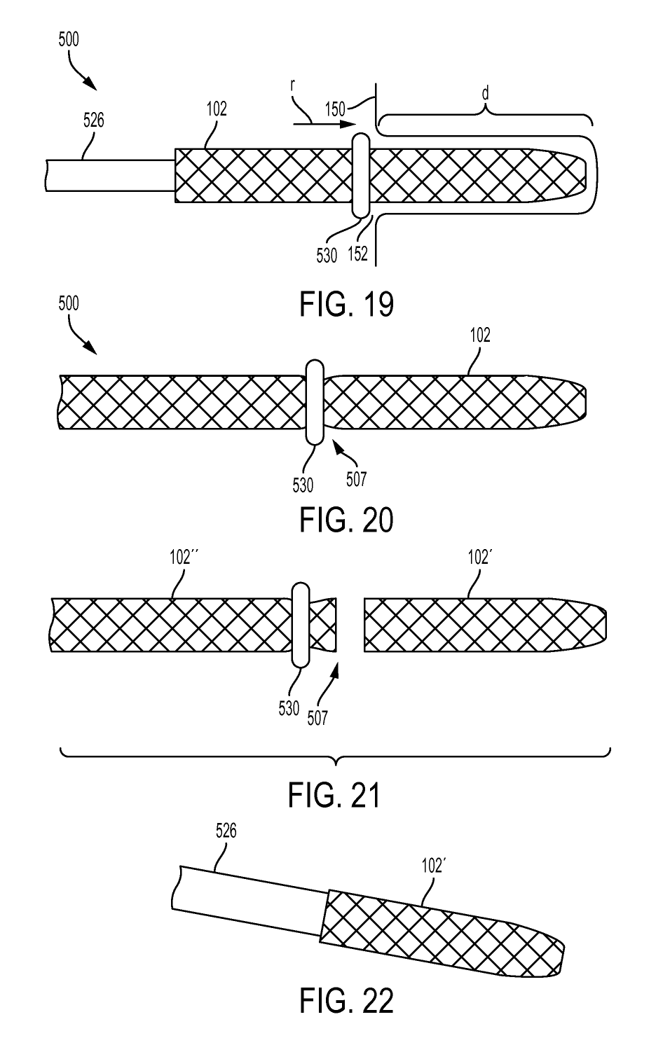

In the embodiment shown in FIGS. 19-22, a system 500 is provided with a hollow tube in the form of an elastomer band 530 is used to hold the woven retention device 102 on a rod 526. Before being cut, the woven retention device 102 and rod 526 may be inserted into the bone hole 152 and the elastomer band 530 placed at a position such that the woven retention device 102 can be cut at the desired cutting position 507. After removing the rod from the bone hole, the elastomer band may optionally be positioned just distally to the distal end of the rod, without moving the elastomer band relative to the woven retention device, so that the woven retention device can be cut through. Then, the woven retention device can be placed on the distal end of the rod, as shown in FIG. 22, to be inserted into the bone.

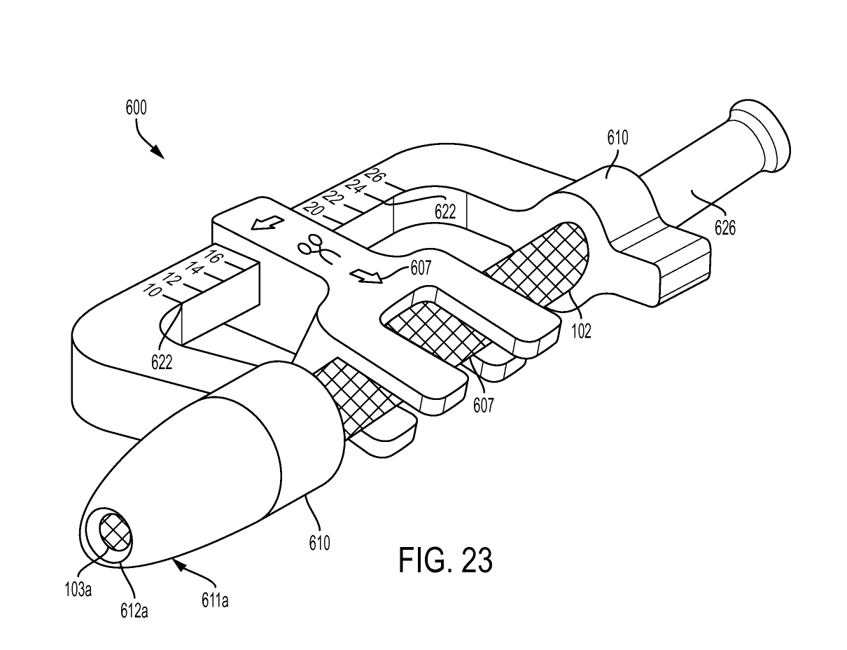

FIG. 23 shows a system 600 including a slidable measuring and cutting device. The device includes a hollow tube 610 that surrounds one or more portions of the woven retention device 102, and that has a part that is slideable relative to the woven retention device 102. Indices 622 indicate a length of the woven retention device 102 distal to a cutting position 607 marked by the arrow on the slidable measuring device. The device also includes a space and indicator for where cutting should be performed by scissors or other cutting implement. After cutting, the push rod 626 may be used to push the woven retention device 102 into the bone through a distal opening 612a at the distal end 611a of the device. In some embodiments, the slideable portion may have an integrated cutting implement.

Next, there are embodiments in which the system incorporates a way of cutting the woven retention device.

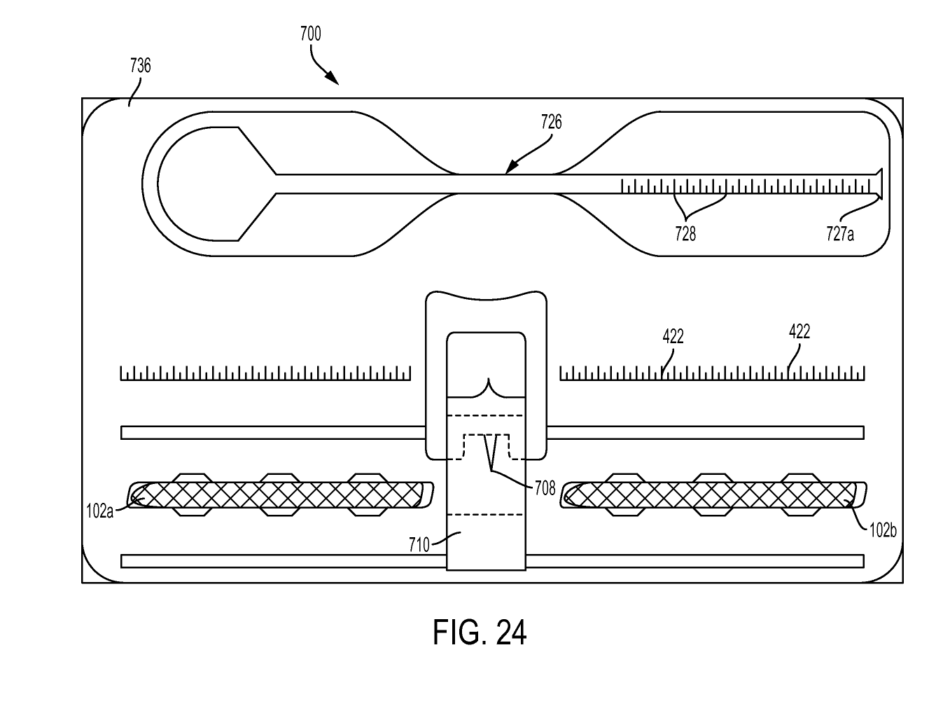

FIG. 24 shows a packaged system 700 including an inserter/depth gauge 726, two woven retention devices 102a, 102b, and a slidable measuring and cutting device 710 incorporated into the packaging 736. The slidable device 710 can be slid to the measurement mark 422 corresponding to the desired length of the woven retention device 102a or 102b. The slidable device 710 may optionally incorporate a cutting implement 708 to shear the woven retention device 102a or 102b when pressed down onto the woven retention device 102a or 102b. The cut woven retention device may then be placed on the inserter 726 for inserting into the bone. In some embodiments, the inserter 726 can be used as a depth gauge to measure the depth of a bone hole by placing the distal end 727a into the bottom of the hole (or rear surface of the bone in a bicortical application) using indices 728 prior to cutting the woven retention devices 102a, 102b.

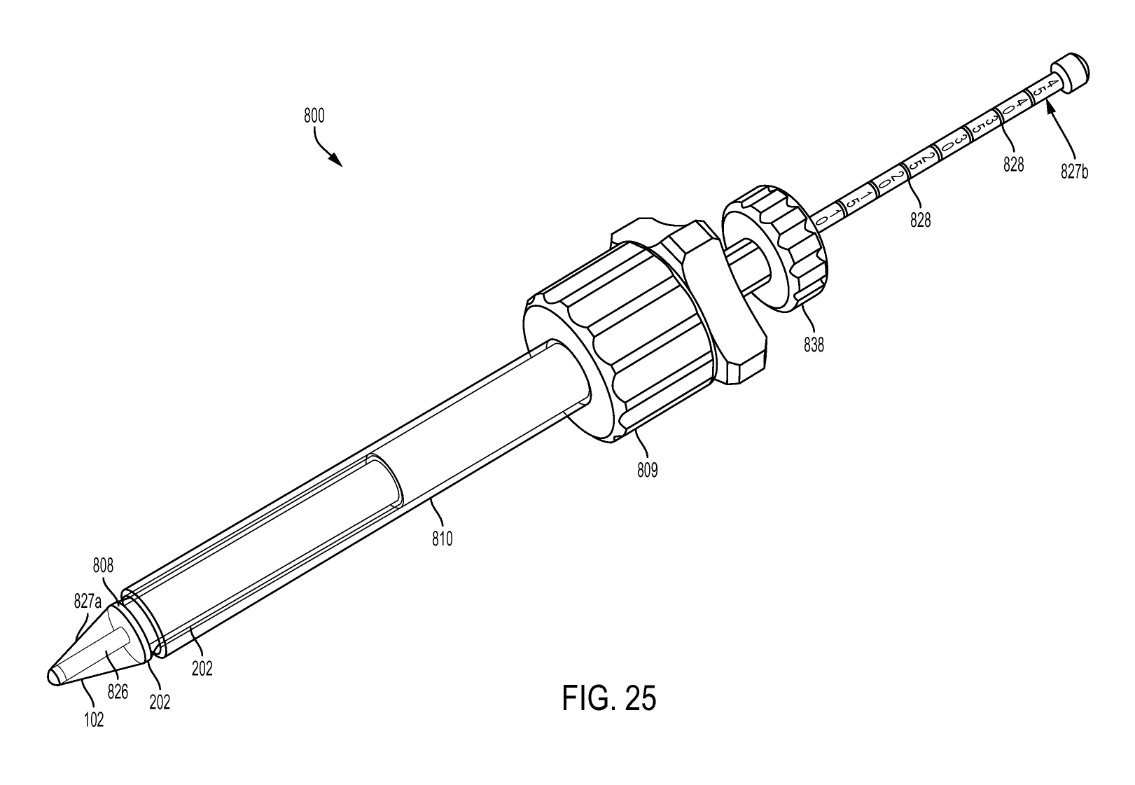

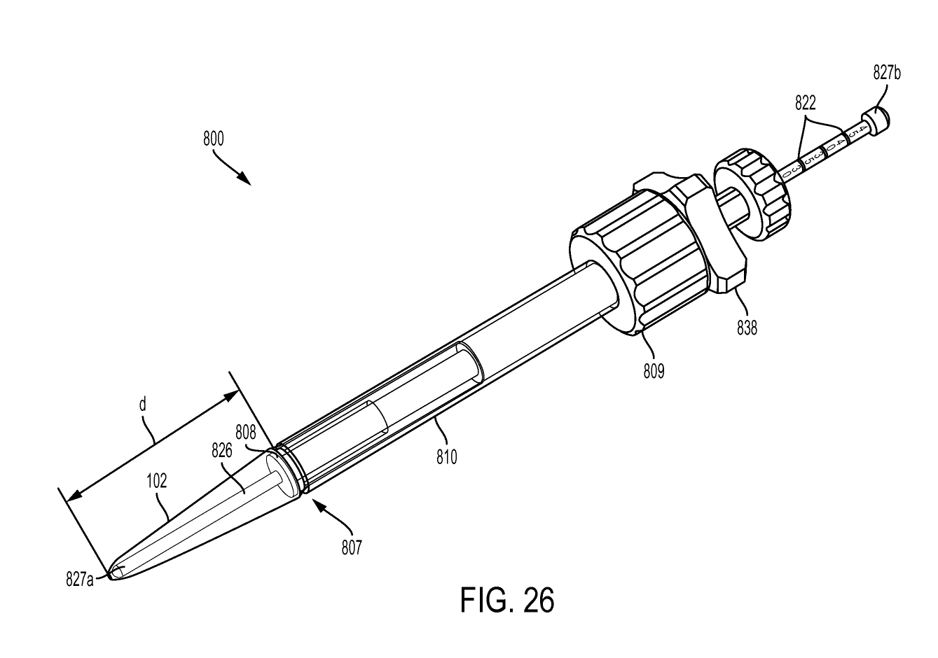

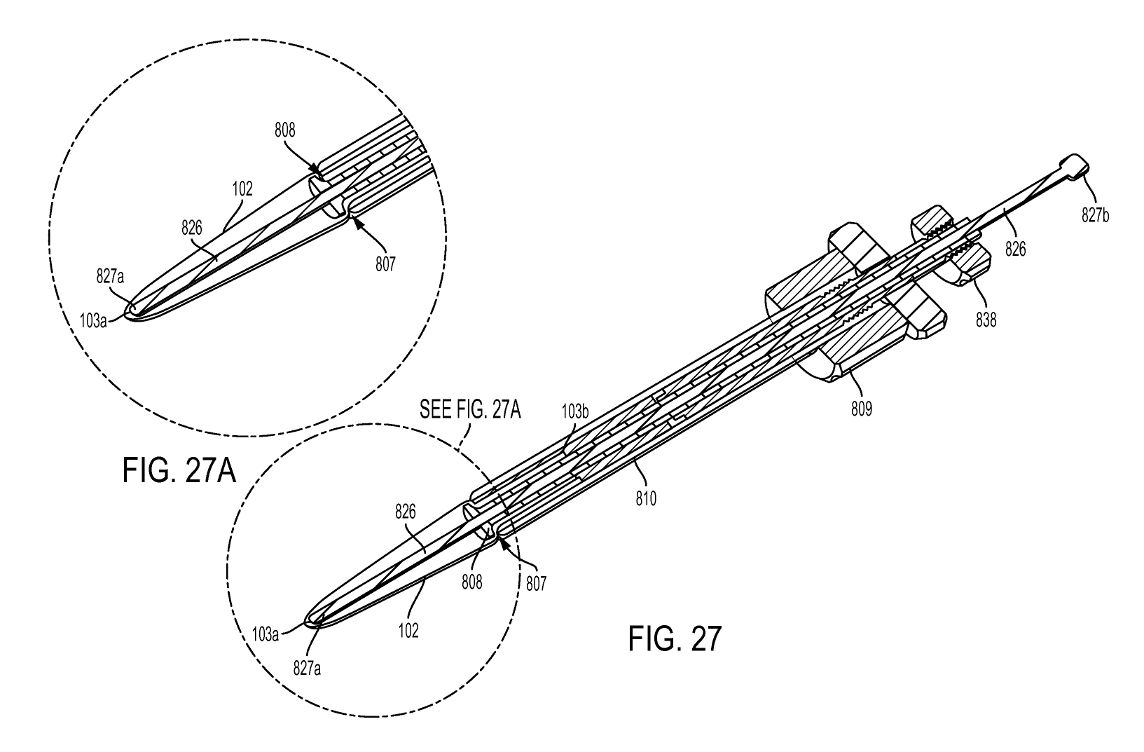

FIGS. 25-27 show a system 800 including a woven retention device shearing tool according to an embodiment. The tool may be provided in the state shown in FIG. 25 and can be pre-loaded with a woven retention device 102, but can also allow for loading or reloading of woven retention devices. The tool includes a punch 808 near the distal end of hollow tube 810, the punch 808 being arranged for punching or shearing a woven retention device 102 to a desired length. The woven retention device 102 is provided on the inside of the tool within the hollow tube 810 and may be advanced distally by advancing the proximal end 827b of push rod 826 until the desired position is reached as indicated by the measuring indices 828 near the proximal end 827b of the push rod 826. Meanwhile, the distal end 827a of the push rod 826 extends the woven retention device distally to until a desired length d of the woven retention device is distal to the punch 808, as shown in FIG. 26. Then, a locking knob 838 may be used to fix the position of the woven retention device 102. A shear knob 809 may then be turned to activate the punch 808 and thus shear the woven retention device 102. FIG. 27 shows a cross-section view of the tool. Although knobs 809 and 838 are used for locking the push rod 826 and for shearing the woven retention device 102, other suitable mechanisms could be substituted.

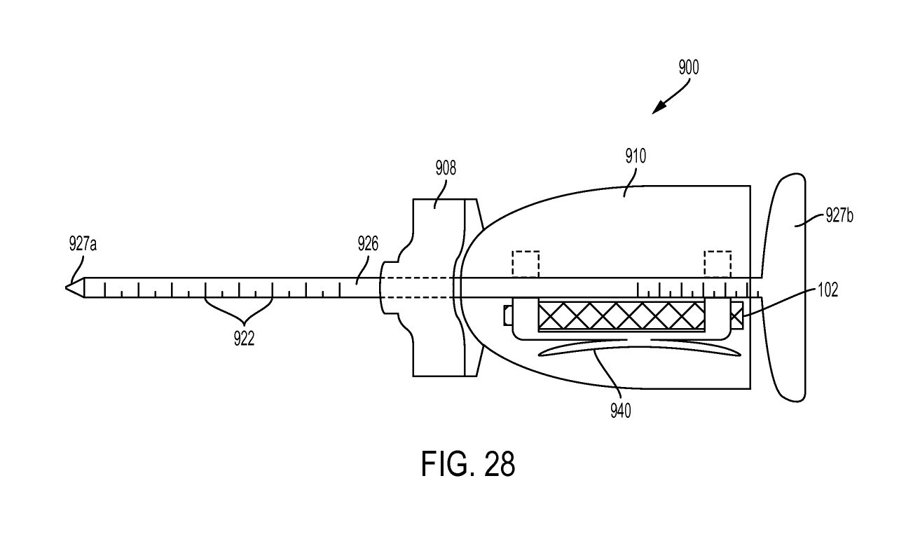

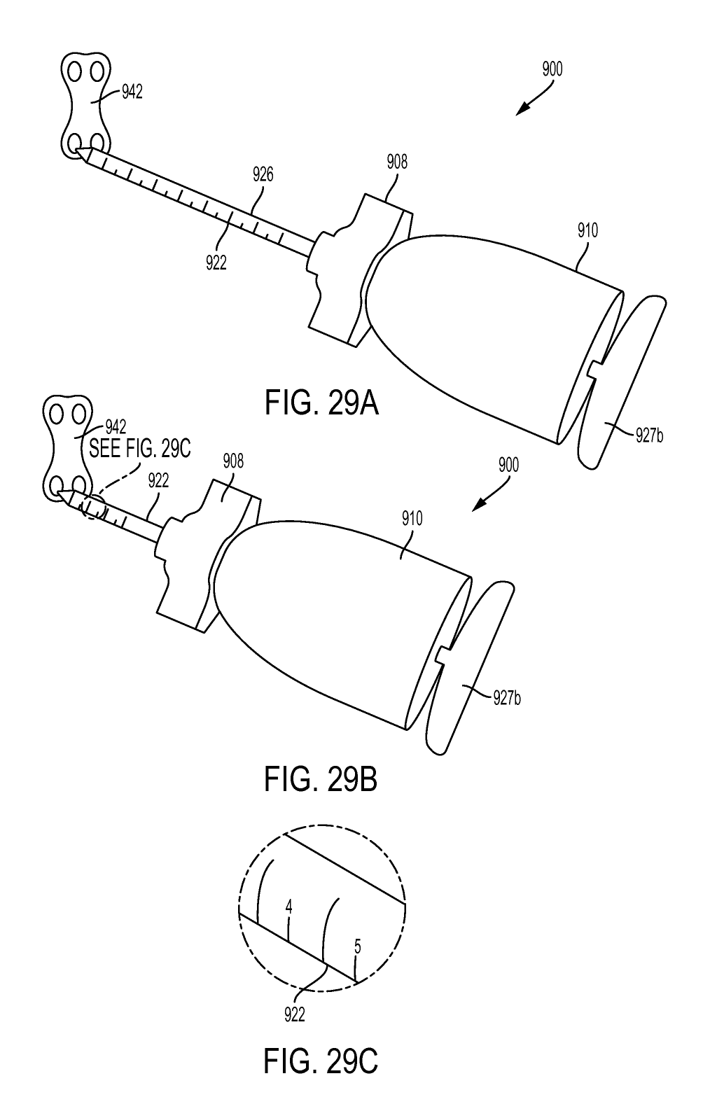

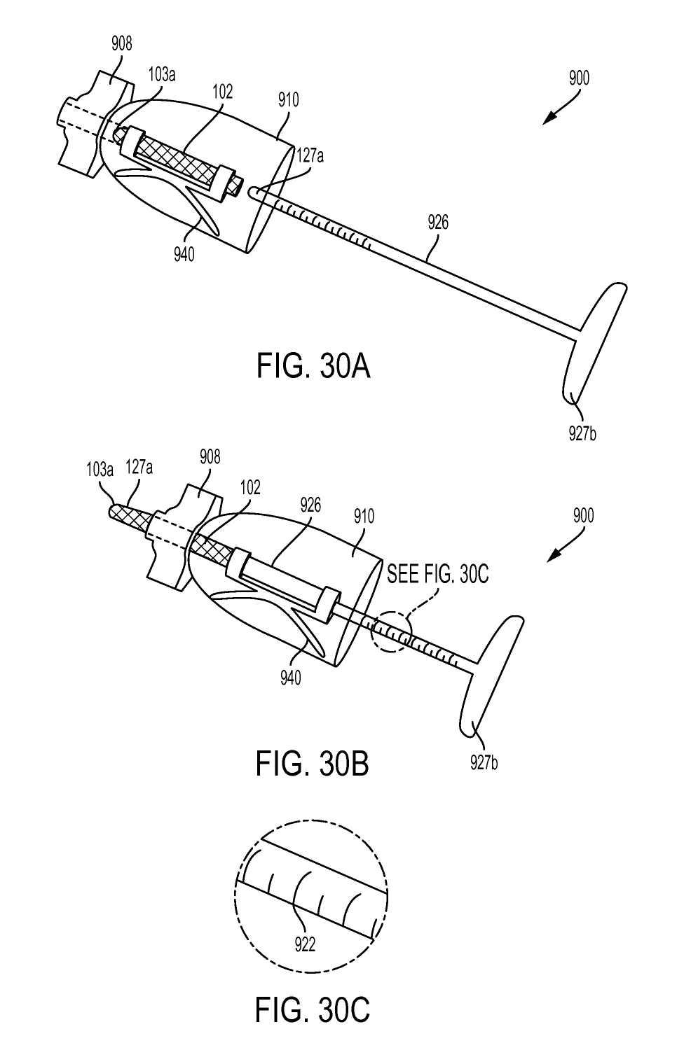

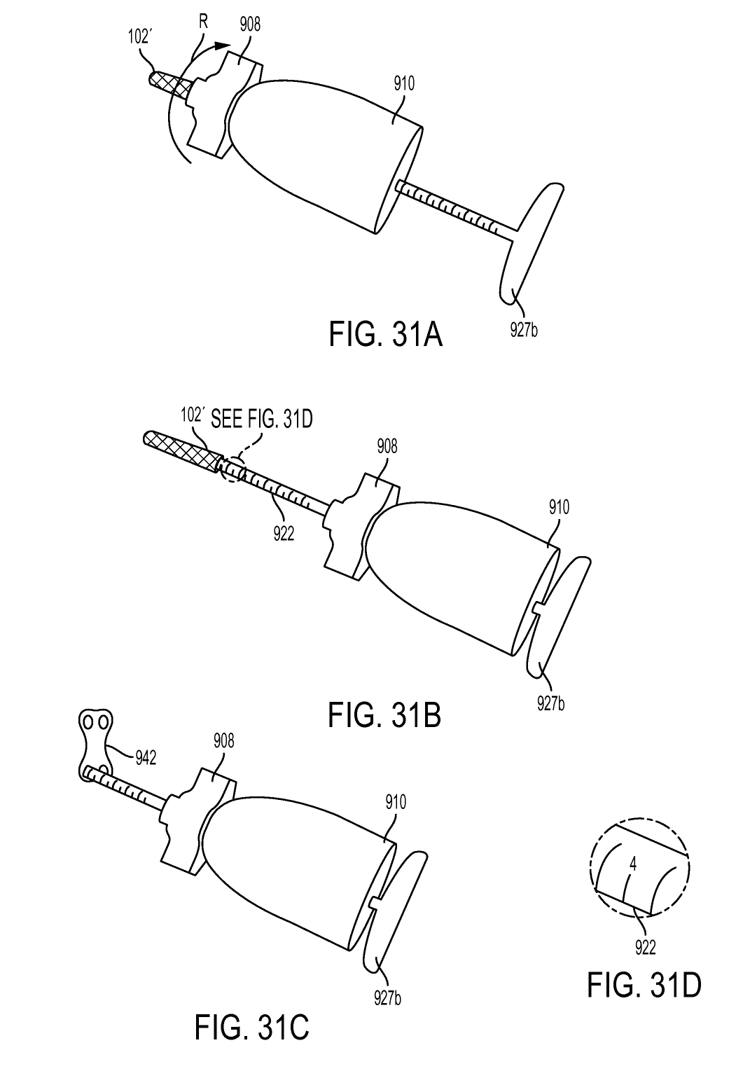

FIG. 28 shows a system 900 according to another embodiment of invention. The system 900 includes a tool 910 with a depth gauge/inserter rod 926 having measuring indices 922. The inserter rod 926 is attached to the plunger 927b on the proximal end. The system 900 also includes a spring-loading mechanism 940 for loading a woven retention device 102 into the tool 910 that surrounds at least a portion of the woven retention device 102, and a rotational cutting element 908. As shown in FIGS. 29A-29C, the depth gauge 926 may first be used to determine a depth of a bone hole (represented by the holes in bone plate 942) using the measuring indices 922. Next, as shown in FIG. 30A, the plunger 927b is retracted, and the woven retention device 102 is spring loaded into the tool 910, as shown in FIG. 30. Then, as shown in FIG. 30B, the plunger 927b is advanced in the distal direction until the desired length of the woven retention device 102 has emerged past the rotational cutting element 908, as indicated by the measuring indices 922 on the depth gauge 926 and/or plunger 927b. As shown in FIG. 31A, the rotational cutting element is then rotated to cut the woven retention device 102. Now measured and cut, the woven retention device 102′ can remain on the inserter rod 926, as shown in FIG. 31B, and subsequently placed into the bone hole using the plunger, as shown in FIG. 31C. The system 900 may be configured to house multiple woven retention devices within the tool 910 so that woven retention devices can be easily reloaded. Alternatively, the spring loading mechanism 940 may be accessible from an exterior of the tool 910 so that additional woven retention devices can be supplied as needed.

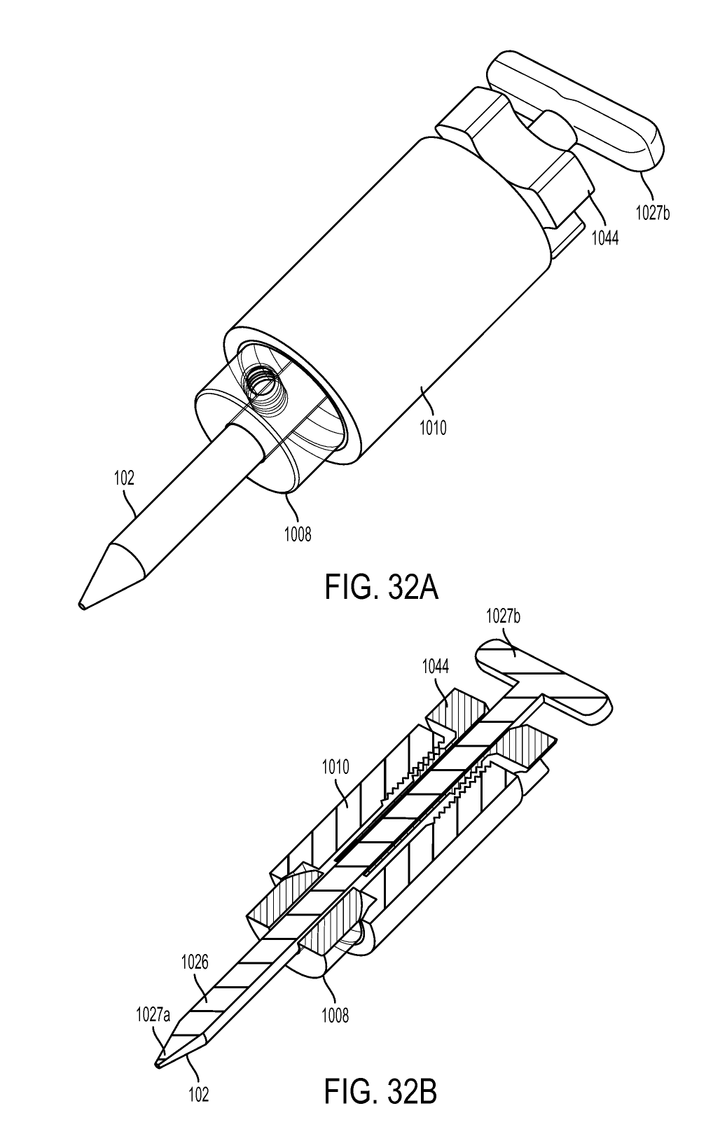

FIGS. 32A-32B show a system 1000 having a tube 1010 at least partially surrounding a rod 1026 and woven retention device 102 placed around the rod 1026. The system 1000 also includes a rotational cutter 1008 to cut the woven retention device 102. The desired length is adjusted by rotating the proximal knob 1044 and then the cut is performed by the rotational cutter 1008.

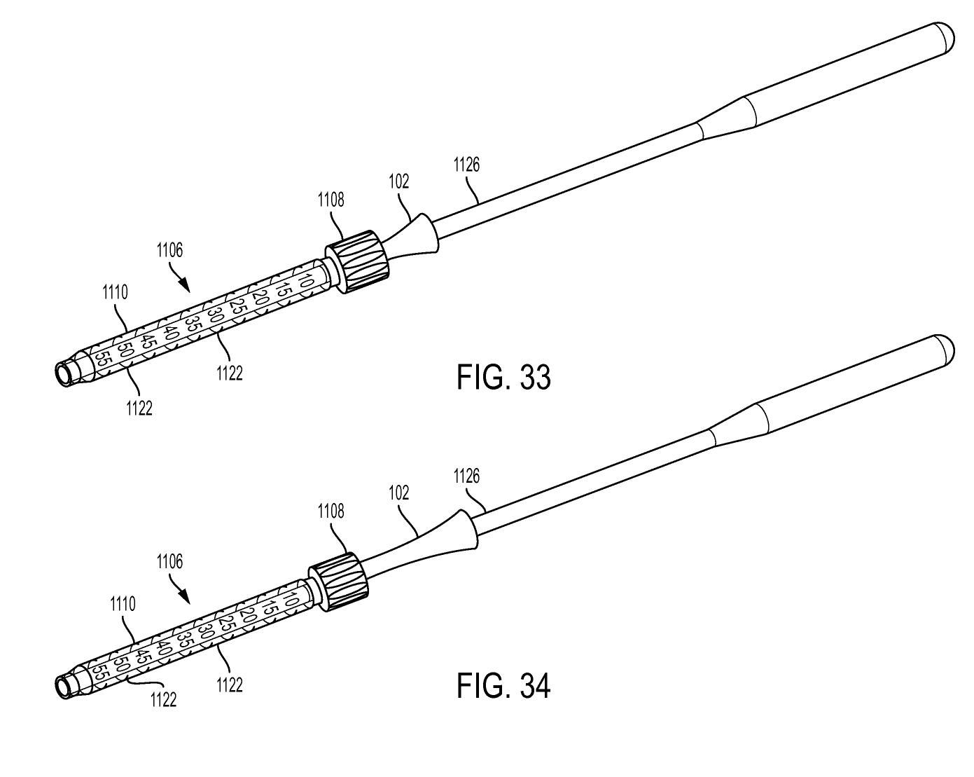

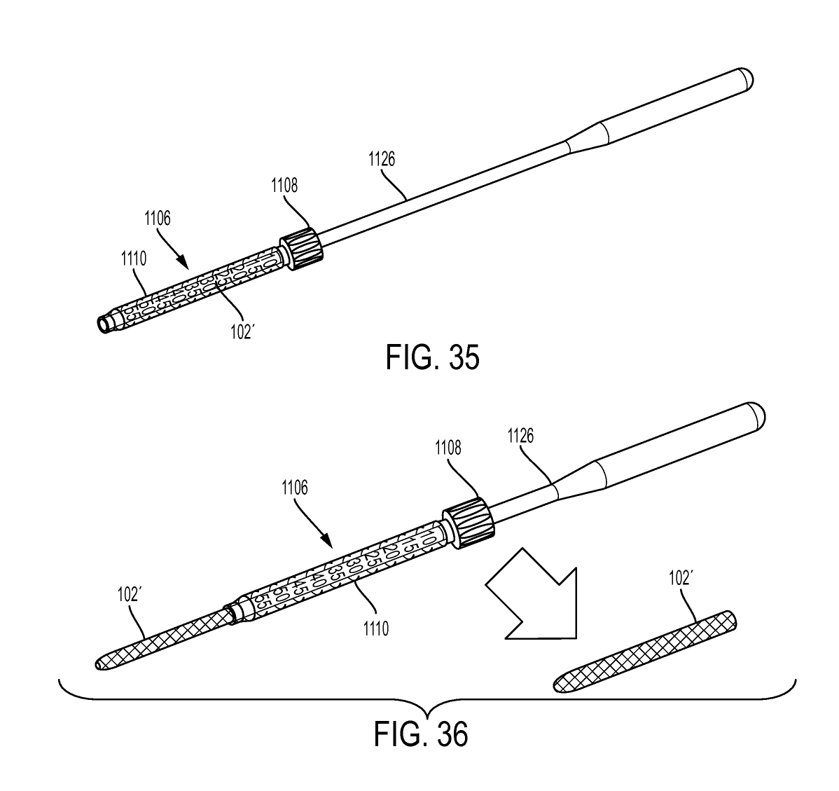

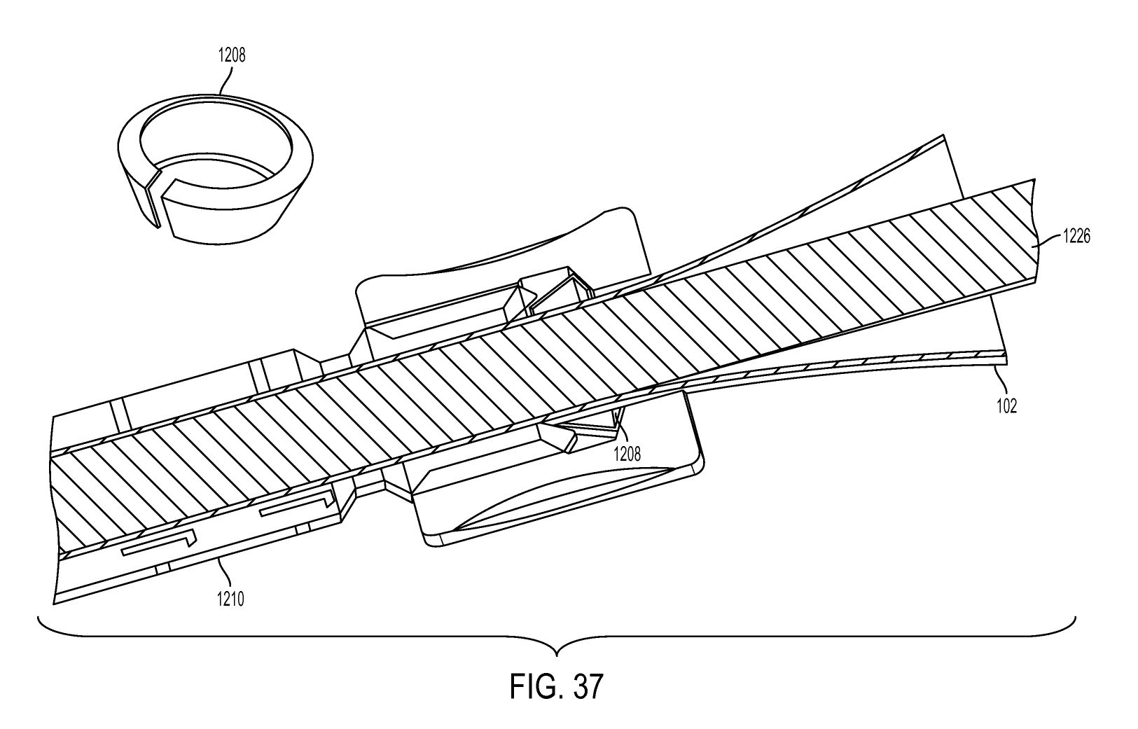

FIGS. 33-37 show a system similar to system 100 in FIGS. 1-6B. However, in FIGS. 33-37, a cutting implement 1108 is provided where the woven retention device 102 enters the hollow tube 1110. A cutting position is located within the cutting implement, and the hollow tube 1110 has reference marks 1122 for measuring a distance from the cutting position to a distal end of the woven retention device, so that the woven retention device can be cut to a desired size. The rod 1126 can be used to place the woven retention device in the desired position for cutting, and can also be used for inserting the woven retention device into a bone hole after being cut to size. In some embodiments, the cutting implement is constructed as shown in FIG. 37, for example, where a cutting edge of the cutting implement 1208 can be pressed into the woven retention device by squeezing an outside of the cutting implement 1208. Optionally, the cutting implement 1208 can be rotated relative to the woven retention device or hollow tube 1210 while squeezing the cutting implement 1208, in order to complete the cutting operation. The configuration of the cutting implement is not limited to the structure shown in FIG. 37, and can take many forms that could be appreciated by a person of ordinary skill in the art.

Alternatively, in some embodiments, a system is provided that can include an inserter gun that can launch the sleeve into the bone hole. The system can also include a balloon dilator that can expand the sleeve once inside the hole and the sleeve stays expanded. The inserter gun may automatically load a woven retention device after firing a previous woven retention device, and may also perform a cutting operation of the woven retention device, according to an embodiment.

The system can include scissors and/or operating room scissors to cut the sleeve or an integral cutting device that guillotines the sleeve, if necessary. The system can further include graduation showing measurements either on the cardboard packaging or the slidable pusher. The system can also include a slidable stop accommodating a prescribed depth to insert.

In addition or alternatively to the above-described cutting of the woven retention device, the woven retention device may be provided in one or more pre-determined lengths corresponding to bone holes of particular sizes.

In some embodiments, a system may be provided with one or multiple woven retention devices. A woven retention device may be packaged with the push rod and delivery tube, and/or packaged separately. Thus, in a case, for example, where at least one of the delivery tube and push rod are re-usable, a packaging can be provided that includes one or more woven retention devices without the delivery tube or push rod.

A woven retention device according to some embodiments may include a proximal end with a proximal opening, a distal end that is tapered to a distal tip, and a sleeve body extending between the proximal and distal ends. The proximal end may be able to receive a fastener through the proximal opening. The delivery tube also includes a proximal end with a proximal opening, a distal end with a distal opening, and a tube body extending between the proximal and distal ends. The push rod may have a proximal end, a distal end with a distal tip, and a rod body.

In an embodiment, a method of using a system for inserting the woven retention device into a bone hole includes pushing the woven retention device through the delivery tube using the push rod. For example, a woven retention device may be pushed through the delivery tube with the push rod. At least part of the push rod may be inserted into the delivery tube through the proximal opening of the delivery tube, and the distal tip of the push rod exerts a force on an interior of the woven retention device in at least the distal end of the woven retention device. It is contemplated that the tip of the push rod may push on a distal-most interior wall of the distal tip and/or on at least a portion of the tapered portion.

In some embodiments, the distal tip of the push rod may be shaped to at least partially conform to a shape of the interior surface of the distal tip of the tapered portion of the woven retention device. For example, the distal tip of the push rod may be tapered or otherwise shaped to contact the inner surface of the woven retention device. In some embodiments, the distal tip of the woven retention devices may be closed, while in other embodiments the distal tip may have an opening with a smaller diameter than the proximal opening of the woven retention device. In a case where the distal end of the woven retention device is open, the diameter at the distal tip may be small enough so that the push rod does not extend through the opening when pushing the woven retention device through the delivery tube and into the bone hole.

The delivery tube may include a compression portion at least at or near the distal end of the delivery tube. The compression portion can force the woven retention device to radially contract as the woven retention device is pushed through the distal opening with the push rod. In some embodiments, the compression portion may be a portion of the delivery tube with an inner diameter that is smaller than an outer diameter of the woven retention device when the woven retention device is in a relaxed state. Further details of the compression portion according to some embodiments will be discussed further below.

According to an embodiment of a method of using a system for inserting a woven retention device into a bone, the distal opening of the delivery tube may be positioned at or near the opening of the bone hole. As such, when the woven retention device is pushed through the delivery tube and is compressed by the compression portion, the woven retention device can easily be inserted into the bone hole due to the radially contracted state of the woven retention device from the compression portion. For example, the contracted state of the woven retention device allows it to easily enter the opening without being obstructed by the periphery of the opening. Also, by staying at least partially contracted even after passing through the compression portion and entering the bone hole, the woven retention device can avoid obstructions in the bone hole itself due to irregularities in the bone hole. According to some embodiments, the woven retention devices have a construction such that they return to their relaxed state after passing fully through the compression portion, the relaxed state being one in which the woven retention device has a diameter that is larger than a diameter when in the compressed state.

It is possible that the size of the bone hole may prevent the woven retention devices from fully returning to their relaxed state if the bone hole has an inner diameter that is smaller than an outer diameter of the woven retention device in the relaxed state. Nonetheless, the woven retention device will be biased to expand or return to the relaxed state. In one embodiment, the woven retention device can elastically expand and/or self-expand to the relaxed state. In expanding towards the relaxed state, the woven retention device will press against the bone surface of the bone hole. It is also possible that the bone hole has an inner diameter that is larger than the outer diameter of the woven retention device in a relaxed state. In such a case, the woven retention device may not fully contact the bone surface of the bone hole when the woven retention device is in the relaxed state. However, the woven retention device may transition to an expanded state when a fastener is later inserted into the woven retention device. Thus, in the expanded state, the woven retention device can provide the desired interaction between the fastener and the bone hole. Therefore, according to the properties of the woven retention device and the compression portion of the delivery tube, a system and method may be provided which allows for easy insertion of the woven retention device into the bone hole while nonetheless ensuring the desired interaction between the woven retention device, the fastener, and the bone surface.

The method according to some embodiments includes inserting a fastener into the woven retention device after the woven retention device is placed into the bone hole. In some embodiments, the fastener is a bone screw. However, embodiments of the invention are not limited to bone screws, and other fasteners such as rods, pins, prosthetic devices, or other devices can be inserted into the woven retention device. The fastener may be inserted fully into the woven retention device such that a proximal end of the fastener is substantially flush with the proximal end of the woven retention device or with the surface of the bone.

According to some embodiments, a method of inserting a woven retention device may include removing an excess portion of the woven retention device so that it fits as desired in a bone hole of a particular size. In this regard, embodiments include systems to aid a user or operator of the woven retention device to easily measure, cut, and insert the woven retention device. The woven retention device is at least partially inserted into a measuring tube that is equipped with a mechanism for measuring the woven retention device. In this example, the measuring is accomplished using measuring indicia on the side of the measuring tube. According to an embodiment of the method, the woven retention device is at least partially inserted into the measuring tube until the distal end of the woven retention device reaches a desired position relative to the measuring indicia. The relative position of the woven retention device can be appreciated by the measuring tube having a clear tube body. After the woven retention device is inserted to the desired position, a portion of the distal end of the woven retention device may be removed using scissors, for example. In an embodiment, the woven retention device is cut at a point approximately level the proximal opening of the measuring tube. Therefore, the measuring indicia can be arranged such that the measuring indicia measure a distance from the proximal opening of the measuring tube and, correspondingly, a length of the woven retention device from the distal end of the woven retention device to proximal opening (or other position where the woven retention device is cut).

According to some embodiments, a portion of the delivery tube near the distal end includes one or more distal slots. The distal slots define one or more prongs at or near the distal end of the delivery tube. The compression portion of the delivery tube can be formed from these compression prongs. For example, the compression prongs are biased radially inward towards a longitudinal axis of the delivery tube, thus forming a narrowed distal opening of the delivery tube.

According to some embodiments, there can be an odd number of distal slots on the distal end of the delivery tube. An odd number can provide more circumferentially uniform compression of the woven retention device because the distal slots are not aligned with each other. An even number of slits would have the slots aligned if the slots are placed symmetrically about the circumference of the delivery tube.

The distal end of the delivery tube, according to an embodiment, may be brought into closer proximity to the bone hole, even to the point of being partially inserted into the bone hole. Insertion of the distal end of the delivery tube can be aided by the tapering of the distal end resulting from the compression prongs and distal slots.

In an embodiment, three distal slots and three compression prongs are formed in the distal end of the delivery tube. Measuring indicia may be provided along the body of delivery tube. The delivery tube also may have a slotted opening (or push guide) formed along a substantial length of the delivery tube. As discussed below, the push guide can assist in pushing the woven retention device through the entirety of the delivery tube until it woven retention device has fully exited the delivery tube into the bone hole. In some embodiments, the push guide is contiguous with the proximal opening at the proximal end of the delivery tube. In other embodiments, the push guide may be separated from the proximal opening on the proximal end. The push guide can also allow an operator of the delivery tube to see a position of the woven retention device through the opening. Thus, the delivery tube need not be made of a transparent material for using the measuring indicia.

In an embodiment, the delivery tube may be outfitted with a push rod having a rod lever on or near the proximal end of the push rod. The rod lever extends radially outward from the rod body through the push guide. An operator of the system can slide the rod lever back and/or forth in the push guide so that the push rod may be easily movable along a substantial length of the delivery tube by an operator of the system. Therefore, the push rod does not need to be long enough for the proximal end of the push rod to remain proximal to the proximal end of the delivery tube because the operator can maintain control of the push rod via the push guide. Thus, a more compact system is provided, saving both space and material costs.

In some embodiments, the woven retention device is fixed to a stopper to which the push rod is coupled. An unseen portion of the push rod may extend past the stopper to push the woven retention device out of the delivery tube. Coupling between the push rod and the stopper is achieved by locking portion. The push rod can be de-coupled from the stopper by a rotation of the push rod about its longitudinal axis. After being decoupled, the push rod can be at least partially retracted from the woven retention device so that a distal portion of the woven retention device can be cut.

The rod lever of the push rod has a substantially planer surface that is perpendicular to the longitudinal axis of the push rod. The compression prongs of the delivery tube can spread apart from one another when the woven retention device is being pushed through them. Thus, the compression prongs can be flexible to allow the woven retention device to pass through the compression prongs while also radially compressing the woven retention device due to the inward bias of the compression prongs.

Some embodiments may include additional systems for packaging, measuring, and preparing the woven retention device for use. Packaging may include a backing card on which the woven retention device is mounted in mounting loops. The backing card may include two cards that are movable relative to one another. The pullout card may be pulled out from the stationary card by pulling the pullout card in the pull tab area. When the pullout card is pulled outwardly from the stationary card, the mounting loop, which is attached to the pullout card, also moves relative to the mounting loop, which is attached to the stationary card. As the pullout card is moved, one or more measuring indices within the measurement windows will move relative to a marker of the stationary card. The measurement indicated by the marker corresponds to a distance between a point on the woven retention device indicated by a marker on the movable mounting loop and the distal tip of the woven retention device that is attached at the attachment portion. Therefore, according to some embodiments, a user of the woven retention device cut the woven retention device to any desired length using the measuring system provided on the packaging of the woven retention device. For example, when the desired length is obtained in the measurement window, the user can cut the woven retention device where indicated by the marker on the movable mounting loop (e.g., a point indicated by the words “cut here,” example). After cutting, the portion of the woven retention device between the attachment portion and the movable mounting loop may be inserted into a bone using other methods described herein.

In some embodiments, multiple measurement windows are provided in the packaging system. Each of the multiple measurement windows may have measuring indices that are calibrated for woven retention devices of a certain type. For example, in an embodiment, the measurement windows may each be calibrated for a woven retention device of a different size, diameter, or weave pattern. In one embodiment, the measurement windows are staggered to calibrate each set of indices to differently constructed or sized woven retention devices. For example, a first measurement window may have measuring indices for a small-diameter woven retention device (e.g., 3.0 mm in diameter). A second window may have measuring indices for a medium-diameter woven retention device (e.g., 5.0 mm in diameter). A third window may have measuring indices for a large-diameter woven retention device (e.g., 9.0 mm in diameter). These diameters are given as examples only. The number of measuring windows provided and the diameters or other properties used to differentiate the measuring scales are not limited to these examples.

According to an embodiment, the packaging may also include mounting portions for a push rod. Delivery tubes may also be included in the packaging, as well as one or more woven retention devices respectively in each packaging. A movable mounting and cutting guide may extend over both woven retention devices so that both can optionally be measured and/or cut at the same time. The distal ends may be arranged on an opposite end of the woven retention devices, so the measuring indicia in measurement windows correspond to a length between the distal ends and the position of the movable mounting and cutting guide.

In some embodiments, the woven retention devices can be packaged within the delivery tubes. This results in compact packaging and, in some embodiments, a woven retention device that can be immediately deployed from the delivery tube. Alternatively, even when packaged within the delivery tube, the woven retention device can be at least partially removed from the delivery tube for measuring and cutting. The push rod may also have a bent portion.

In the packaging systems according to some embodiments, a delivery tube is packaged with a straight push rod. The delivery tubes and push rods may be reusable, while the woven retention devices are not reusable because they are implanted into the patient. Thus, it is contemplated that, in some embodiments, the woven retention device can be packaged separately from the delivery tube and/or push rod. Alternatively, the woven retention device can be packaged with one or both of the delivery tube and the push rod. When the woven retention device is included in the package with the delivery tube, the woven retention device can be pre-inserted into the delivery tube. By packaging the woven retention device within the delivery tube, a user of the system can avoid the step of inserting the woven retention device into the delivery tube.

Multiple measurement windows on the packaging or delivery tube may be used for a different measuring scale corresponding to a woven retention device having different diameters, for example.

Packaging systems may include delivery tubes and push rods, as well as woven retention devices that are pre-loading in the delivery tubes. Because no push guide is providing along the side wall of the delivery tubes, the push rods in the embodiment must be long enough to at least extend from one side of the delivery tubes to the other side in order to push the woven retention devices out of the delivery tubes and into a bone of a patient. These long push rods result in a larger overall package.

In contrast, according to an embodiment, a delivery tube with a push guide can provide an overall shorter package and push rod that is shorter in length. The push rod does not necessarily have to be longer than the delivery tube, because the push guide allows the push rod to slide down the length of the delivery tube. The bent portion of the push rod allows the handle to remain outside of the delivery tube while the distal end of the push rod pushes the woven retention device inside the delivery tube.

In some embodiments, the push guide may extend over only part of the length of the delivery tube. In this way, the push rod may be prevented from all the way through the distal opening of the delivery tube. However, it is possible that the push guide may extend over the entire length of the delivery tube in some embodiments. The length of the push guide may be coordinated with the placement and length of the measuring indicia so that the woven retention device can be measured using the entire range of the measuring indicia based on the distance the woven retention device can be pushed by the push rod.

During the pushing of the woven retention device through the delivery tube, due to the bent portion of the push rod and the push guide, the handle can extend beyond the proximal opening of the delivery tube, on an outside of the delivery tube. Thus, embodiments may provide a compact and sufficient system and method of inserting a woven retention device into a bone.

The foregoing descriptions include examples of embodiments of the woven retention devices and tools for using and inserting the woven retention devices. However, variations in these devices and system are contemplated within the scope of the invention. For example, in some embodiments, the distal tip of the woven retention devices may be closed, while in other embodiments the distal tip may have an opening with a smaller diameter than the proximal opening of the woven retention device.

### EXAMPLES

The following is provided as an example of sizes and dimensions of a delivery tube and push rod according to an example of one embodiment of the invention. However, embodiments are not limited to the following dimensions.

The push rod may be formed of stainless steel (e.g., 316 SS). In an embodiment where the push rod is formed without the bent portion, the push rod may have an overall length of 300 mm, where a handle portion of the push rod may be about 50 mm long and the elongated arm that is inserted into the delivery tube and woven retention device may be 250 mm. In an embodiment where the push rod is formed with the bent portion, the overall length may be shorter. The elongated are may have a diameter of about 2 mm. The diameter of the elongated arm may be smaller or larger, as long as the elongated arm is able to slide within the delivery tube and push the woven retention device. The handle portion may have a diameter of 8 mm, for example.

The delivery tube may have an overall length of about 180 mm. If a push guide is formed in the side of the delivery tube, then the push guide may extend for about 160 mm along the length of the delivery tube. A length of the compression prongs and end slots formed in the delivery tube may be about 15 mm. The outer diameter of the delivery tube may be about 8.5 mm, and an inner diameter may be about 7 mm. The distal opening formed at the tip of the compression prongs may have a diameter of 2.5 mm. This diameter may expand as the woven retention device is pushed through the flexible compression prongs, in some embodiments. The measurement scale of the measuring indices on the delivery tube may be spaced about 0.5 mm apart, in one example.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

1. A system for preparing and inserting a woven retention device into bone, the system comprising:
a woven retention device having a distal end, a proximal end configured to receive a fastener, and a sleeve body between the distal and proximal ends, the sleeve body having a longitudinal axis extending between the distal and proximal ends; and
a measuring device at least partially surrounding a portion of the woven retention device and defining a cutting position along the longitudinal axis, the cutting position being a position at which the woven retention device is to be cut for insertion into a hole in the bone, the measuring device arranged to measure a distance from the distal end to the cutting position,
wherein the woven retention device is movable relative to a least a portion of the measuring device that defines the cutting position such that the woven retention device can be cut to a desired size.

2. The system according to claim 1, further comprising a cutting implement configured to cut the woven retention device at the cutting position.

3. The system according to claim 2, wherein the cutting implement is coupled to the measuring device.

4. The system according to claim 3, wherein the measuring device comprises a hollow tube with a distal end and a proximal end, and an opening on the proximal end or the distal end of the hollow tube, the hollow tube being sized to surround at least a portion of the woven retention device, and the cutting implement being integral with an interior of the hollow tube.

5. The system according to claim 1, wherein the measuring device comprises a hollow tube with an opening on a proximal end or a distal end of the hollow tube, the hollow tube being sized to surround at least a portion of the woven retention device.

6. The system according to claim 5, wherein the cutting position is located at the proximal end or the distal end of the hollow tube.

7. The system according to claim 5, wherein the hollow tube comprises an opening around at least a portion of an outer circumferential surface of the hollow tube, the opening being located between the proximal and distal ends and defining the cutting position.

8. The system according to claim 7, wherein the opening around the portion of the outer circumferential surface of the hollow tube is configured to receive a cutting implement to cut the woven retention device.

9. The system according to claim 8, further comprising the cutting implement, the cutting implement having a cutting edge configured to cut the woven retention device through the opening.

10. The system according to claim 9, wherein the cutting implement is rotatable relative to the woven retention device and is configured to cut the woven retention device while rotating relative to the woven retention device.

11. The system according to claim 5, wherein the measuring device comprises one or more reference marks at fixed distances from the proximal or distal opening of the hollow tube, the one or more references marks indicating the distance from the distal end of the woven retention device to the cutting position.

12. The system according to claim 11, wherein the hollow tube comprises an opening or transparent portion on a circumferential wall of the hollow tube such that a position of the distal end of the woven retention device within the hollow tube relative to the one or more reference marks is visible to a user of the system from an exterior of the hollow tube.

13. The system according to claim 5, further comprising a push rod configured to be slideably received within the hollow tube and the woven retention device, and having a distal end configured to push the woven retention device through the distal opening of the hollow tube.

14. The system according to claim 13, wherein the push rod comprises one or more reference marks at predetermined distances from the distal end of the push rod, the reference marks being arranged to indicate the distance from the distal end of the woven retention device to the cutting position based on a relative position of the reference marks and the measuring device in a state where the push rod is slideably received within the hollow tube.

15. The system according to claim 1, wherein the measuring device comprises a flexible band configured to surround the woven retention device at the cutting position.

16. The system according to claim 15, further comprising a rod for the woven retention device, the rod having a handle portion and a mounting portion, the mounting portion being configured to be inserted within the woven retention device through the proximal end,
wherein the rod is configured to position the woven retention device, while mounted on the mounting portion, into the hole in the bone while the handle portion is held be a user of the system, and
wherein the flexible band is configured to be placed at the cutting position on the woven retention device while the woven retention device is mounted on the mounting portion and within the hole in the bone, the cutting position being approximately at an opening of the hole.

17. An apparatus for measuring and cutting a woven retention device for insertion into a bone, comprising:
a hollow tube having a distal end with a distal opening and a proximal end with a proximal opening, the hollow tube being sized to contain the woven retention device in an interior of the hollow tube, and the distal opening being sized to pass the woven retention device therethrough;
a cutting implement configured to cut the woven retention device to a desired length while at least a portion of the woven retention device is within the hollow tube; and
an advancement mechanism comprising a proximal rod that protrudes from the proximal end of the hollow tube, and a distal end configured to engage the woven retention device within the hollow tube, the advancement mechanism being configured to change a position of the woven retention device within the hollow tube by sliding the woven retention device along a longitudinal axis of the hollow tube to increase or decrease a distance between a distal end of the woven retention device and the cutting implement,
wherein the advancement mechanism has reference marks indicating the distance from the distal end of the woven retention device to the cutting implement.

18. The apparatus according to claim 17, wherein the advancement mechanism further comprising a user-operable lock for fixing a position of the distal end of the advancement mechanism.
